SEQUENCE LISTING (20 sequences)

SEQ ID NO: 1 - protein, 5 aa, Artificial Sequence
  HCDR1, EGFR binding arm
TYGMH

SEQ ID NO: 2 - protein, 17 aa, Artificial Sequence
  HCDR2, EGFR binding arm
VIWDDGSYKYYGDSVKG

SEQ ID NO: 3 - protein, 16 aa, Artificial Sequence
  HCDR3, EGFR binding arm
DGITMVRGVMKDYFDY

SEQ ID NO: 4 - protein, 11 aa, Artificial Sequence
  LCDR1, EGFR binding arm
RASQDISSALV

SEQ ID NO: 5 - protein, 7 aa, Artificial Sequence
  LCDR2, EGFR binding arm
DASSLES

SEQ ID NO: 6 - protein, 9 aa, Artificial Sequence
  LCDR3, EGFR binding arm
QQFNSYPLT

SEQ ID NO: 7 - protein, 5 aa, Artificial Sequence
  HCDR1, c-Met binding arm
SYGIS

SEQ ID NO: 8 - protein, 17 aa, Artificial Sequence
  HCDR2, c-Met binding arm
WISAYNGYTNYAQKLQG

SEQ ID NO: 9 - protein, 10 aa, Artificial Sequence
  HCDR3, c-Met binding arm
DLRGTNYFDY

SEQ ID NO: 10 - protein, 11 aa, Artificial Sequence
  LCDR1, c-Met binding arm
RASQGISNWLA

SEQ ID NO: 11 - protein, 7 aa, Artificial Sequence
  LCDR2, c-Met binding arm
AASSLLS

SEQ ID NO: 12 - protein, 9 aa, Artificial Sequence
  LCDR3, c-Met binding arm
QQANSFPIT

SEQ ID NO: 13 - protein, 125 aa, Artificial Sequence [sequence omitted]

SEQ ID NO: 14 - protein, 107 aa, Artificial Sequence [sequence omitted]

SEQ ID NO: 15 - protein, 119 aa, Artificial Sequence
  VH, c-Met binding arm
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Artificial Sequence
  VL, c-Met binding arm
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Artificial Sequence
  Heavy chain 1
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Artificial Sequence
  Light chain 1
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Artificial Sequence
  Heavy chain 2
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Artificial Sequence
  Light chain 2
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0064307 A1
(43) Pub. Date: Mar. 3, 2022

(54) Stable Formulations Comprising A Bispecific EGFR/C-Met Antibody

(71) Applicant: Janssen Biotech, Inc., Horsham, PA (US)

(72) Inventors: Jessica Kupec, Quakertown, PA (US); Melissa Schreyer, West Hartford, CT (US); Patrick Stahl, Exton, PA (US); Satyen Torne, Chesterbrook, PA (US)

(21) Appl. No.: 17/412,346

(22) Filed: Aug. 26, 2021

Related U.S. Application Data
(60) Provisional application No. 63/070,440, filed on Aug. 26, 2020.

(51) Int. Cl. C07K 16/28; A61K 47/26; A61K 47/20; A61K 47/18; A61K 47/22; A61J 1/14

(52) U.S. Cl. CPC C07K 16/2863; A61K 47/26; A61K 47/20; A61K 47/183; C07K 2317/31; A61J 1/1406; C07K 2317/56; C07K 2317/565; A61K 47/22

(57) ABSTRACT

Provided herein are stable aqueous pharmaceutical compositions comprising formulations of a bispecific epidermal growth factor receptor (EGFR)/hepatocyte growth factor receptor (c-Met) antibody and methods of preparing the same. Also provided herein are methods of treating cancer in a subject in need thereof by administering to the subject the stable aqueous pharmaceutical compositions as disclosed herein. Further provided herein are kits and articles of manufacture comprising the stable aqueous pharmaceutical compositions as disclosed herein.

### CROSS-REFERENCE TO RELATED APPLICATIONS

This application claims priority to U.S. Provisional Application Ser. No. 63/070,440, filed 26 Aug. 2020. The entire contents of the aforementioned application are incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “JBI6337WOPCT1SEQLIST.txt”, creation date of Jul. 26, 2021 and having a size of 19 KB. The sequence listing submitted via EFS-Web is part of the specification and is herein incorporated by reference in its entirety.

### FIELD OF THE INVENTION

Disclosed are compositions and methods for formulating a stable pharmaceutical composition comprising bispecific EGFR/c-Met antibodies.

### BACKGROUND OF THE INVENTION

The role of both epidermal growth factor receptor (EGFR, ErbB1 or HER1) and hepatocyte growth factor receptor (c-Met) in cancer is well established, making these targets attractive for combination therapy. Both receptors signal through the same survival and anti-apoptotic pathways (ERK and AKT). Combination therapies targeting EGFR and c-Met or bispecific anti-EGFR/c-Met molecules have been tested in various clinical trials. While bispecific anti-EGFR/c-Met antibodies have shown promising results, there remains a need in the art for pharmaceutical compositions comprising such antibodies that are stable for long periods of time at refrigerated (2-8° C.) and ambient temperatures.

### SUMMARY OF THE INVENTION

Disclosed herein are stable aqueous pharmaceutical compositions comprising specific formulations of a bispecific antibody.

In one aspect, provided herein are stable aqueous pharmaceutical compositions comprising:
a) about 44 mg/mL to about 56 mg/mL of a bispecific epidermal growth factor receptor (EGFR)/hepatocyte growth factor receptor (c-Met) antibody, the bispecific antibody comprising:
   a first heavy chain (HC1) comprising a HC1 variable region 1 (VH1);
   a first light chain (LC1) comprising a light chain variable region 1 (VL1);
   a second heavy chain (HC2) comprising a HC2 variable region 2 (VH2); and
   a second light chain (LC2) comprising a light chain variable region 2 (VL2),
   wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively, the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively;
b) about 8 mM to about 12 mM of histidine and/or pharmaceutically acceptable histidine salt,
c) about 6.8% (w/v) to about 10.2% (w/v) of sucrose,
d) about 0.036% (w/v) to about 0.084% (w/v) of polysorbate 80 (PS80),
e) about to 0.8 mg/mL to about 1.2 mg/mL of methionine,
f) about 16 μg/mL to about 24 μg/mL of ethylenediaminetetraacetic acid (EDTA); and
g) a pH from about 5.2 to about 6.2.

Provided herein are also methods of treating cancer in a subject in need thereof. The methods comprise administering to the subject the stable aqueous pharmaceutical compositions as disclosed herein.

Also, provided herein are methods for preparing stable aqueous pharmaceutical compositions of a bispecific antibody targeting EGFR and cMet comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively. The methods comprise combining a composition comprising about 50 mg/mL of the bispecific antibody, about 10 mM histidine and/or pharmaceutically acceptable histidine salt, about 8.5% Sucrose, and about 1 mg/mL L-methionine with polysorbate 80 to a final concentration of about 0.06% (w/v) and EDTA to a final concentration of about 20 μg/mL, wherein the stable aqueous pharmaceutical composition has about pH 5.7.

Also, provided herein are kits comprising the stable pharmaceutical aqueous formulations as disclosed herein and instructions for use thereof.

Further provided herein are articles of manufacture comprising a container holding the stable aqueous pharmaceutical formulations as disclosed herein.

### DETAILED DESCRIPTION OF THE INVENTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.”

The term “antibody,” and like terms is meant in a broad sense and includes immunoglobulin molecules or fragments thereof, including monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full-length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and (constant heavy 1) CH1 domains; a F(ab)₂ fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; and a domain antibody (dAb) fragment (Ward et al., Nature 341:544-546, 1989), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed combination therapy effective, at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer (e.g. non-small cell lung cancer (NSCLC)) and the like.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

### DESCRIPTION

Disclosed herein are stable, aqueous pharmaceutical compositions comprising a bispecific EGFR/c-Met antibody.

In some aspects, the stable aqueous pharmaceutical composition comprises a bispecific EGFR/c-Met antibody comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively; the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively (see table 14). The stable aqueous pharmaceutical composition also comprises a histidine and/or pharmaceutically acceptable histidine salt, sucrose, polysorbate 80 (PS80), methionine, and ethylenediaminetetraacetic acid (EDTA), and a pH from about 5.2 to about 6.2. The stable aqueous pharmaceutical compositions provided herein further comprise about 44 mg/mL to about 56 mg/mL of the bispecific EGFR-cMet antibody, about 8 mM to about 12 mM of histidine and/or pharmaceutically acceptable histidine salt, about 6.8% (w/v) to about 10.2% (w/v) of sucrose, about 0.036% (w/v) to about 0.084% (w/v) of polysorbate 80 (PS80), about to 0.8 mg/mL to about 1.2 mg/mL of methionine, about 16 μg/mL to about 24 μg/mL of EDTA; and a pH from about 5.2 to about 6.2.

In some embodiments, the first heavy chain (HC1) of the bispecific EGFR-cMet antibody comprises a HC1 constant domain 3 (HC1 CH3) and a HC1 variable region 1 (VH1). In some embodiments, the second heavy chain (HC2) of the bispecific EGFR-cMet antibody comprises a HC2 constant domain 3 (HC2 CH3) and a HC2 variable region 2 (VH2). In some embodiments, the first heavy chain (HC1) of the bispecific EGFR-cMet antibody comprises a HC1 constant domain 3 (HC1 CH3) and a HC1 variable region 1 (VH1 and the second heavy chain (HC2) of the bispecific EGFR-cMet antibody comprises a HC2 constant domain 3 (HC2 CH3) and a HC2 variable region 2 (VH2). In some embodiments, the first heavy chain (HC1) of the bispecific EGFR-cMet antibody comprises a HC1 constant domain 2 and constant domain 3 (HC1 CH2-CH3) and a HC1 variable region 1 (VH1). In some embodiments, the second heavy chain (HC2) of the bispecific EGFR-cMet antibody comprises a HC2 constant domain 2 and constant domain 3 (HC2 CH2-CH3) and a HC2 variable region 2 (VH2). In some embodiments, the first heavy chain (HC1) of the bispecific EGFR-cMet antibody comprises a HC1 constant domain 2 and constant domain 3 (HC1 CH2-CH3) and a HC1 variable region 1 (VH1) and the second heavy chain (HC2) of the bispecific EGFR-cMet antibody comprises a HC2 constant domain 2 and constant domain 3 (HC2 CH2-CH3) and a HC2 variable region 2 (VH2).

In some embodiments, the bispecific antibody comprises asymmetric stabilizing mutations in the HC1 CH2-CH3 region, in the HC2 CH2-CH3 region, or both. “Asymmetric stabilizing mutations” refers to mutations in a first CH2-CH3 region and in a second CH2-CH3 region which are at different positions in the first and in the second CH2-CH3 region and favor (e.g. stabilize) heterodimer formation between the first CH2-CH3 region and the second CH2-CH3 region over homodimer formation between the first CH2-CH3 region or the second CH2-CH3 region. Exemplary asymmetric stabilizing mutations in the HC1 CH2-CH3 region and the HC2 CH2-CH3 region, or in the HC2 CH2-CH3 region and the HC1 CH2-CH3 region, are (wherein residue numbering is according to the EU Index):
F405L and K409R, respectively;
wild-type and F405L/R409K, respectively;
T366W and T366S/L368A/Y407V, respectively;
T366Y/F405A and T394W/Y407T, respectively;
T366W/F405W and T394S/Y407A, respectively;
F405W/Y407A and T366W/T394S, respectively;
L351Y/F405A/Y407V and T394W, respectively;
T366I/K392M/T394W and F405A/Y407V, respectively;
T366L/K392M/T394W and F405A/Y407V, respectively;
L351Y/Y407A and T366A/K409F, respectively;
L351Y/Y407A and T366V/K409F, respectively;
Y407A and T366A/K409F, respectively;
D399K/E356K and K409D/K392D, respectively; or
D399K/E356K/E357K and K409D/K392D/K370, respectively.

In some embodiments, the bispecific EGFR-cMet antibody comprises an HC1 variable region comprising the amino acid sequence of SEQ ID NO:13 and a LC1 variable region comprising the amino acid sequence of SEQ ID NO:14 (see table 14). In some embodiments, the bispecific antibody comprises asymmetric stabilizing mutations in the HC1 CH2-CH3 region, in the HC2 CH2-CH3 region, or both. In some embodiments, the bispecific antibody comprises K409R in the c-Met binding arm and F405L in the EGFR binding arm.

In some aspects, the bispecific EGFR-cMet antibody comprises a HC2 variable region comprising the amino acid sequence of SEQ ID NO:15 and a LC2 variable region comprising the amino acid sequence of SEQ ID NO:16 (see table 14).

In some embodiments, the heavy chain 1 (HC1) comprises the amino acid sequence of SEQ ID NO:17 and the HC2 comprises the amino acid sequence of SEQ ID NO:19 (see table 14).

In some embodiments, the light chain 1 (LC1) comprises the amino acid sequence of SEQ ID NO:18 and the LC2 comprises the amino acid sequence of SEQ ID NO:20 (see table 14).

In some embodiments, bispecific EGFR-cMet antibody is amivantamab.

According to some aspects, the stable aqueous pharmaceutical composition comprises the bispecific EGFR-cMet antibody at a concentration of about: 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, or 65 mg/mL. In some embodiments, the bispecific EGFR-cMet antibody has a concentration of about 50 mg/mL.

According to some aspects, the stable aqueous pharmaceutical composition comprises histidine and/or pharmaceutically acceptable histidine salt at a concentration of about: 8 mM, 9 mM, 10 mM, 11 mM, 12 mM, 13 mM, 14 mM, or 15 mM. In some embodiments, the histidine and/or pharmaceutically acceptable histidine salts has a concentration of about 10 mM. In a further embodiment, the histidine and/or pharmaceutically acceptable histidine salt comprises L-histidine and L-histidine hydrochloride monohydrate.

According to some aspects, the stable aqueous pharmaceutical composition comprises sucrose at a concentration (percentage of weight to volume (% w/v)) of about: 6.0%, 6.1%, 6.2%, 6.3%, 6.4%, 6.5%, 6.6%, 6.7% 6.8%, 6.9%, 7.0%, 7.1%, 7.2%, 7.3%, 7.4%, 7.5%, 7.6%, 7.7% 7.8%, 7.9%, 8.0%, 8.1%, 8.2%, 8.3%, 8.4%, 8.5%, 8.6%, 8.7% 8.8%, 9.9%, 10.0%, 10.1%, 10.2%, 10.3%, 10.4%, 10.5%, 10.6%, 10.7% 10.8%, 10.9%, or 11.0%. In some embodiments, the stable aqueous pharmaceutical composition comprises about 8.5% (w/v) sucrose.

According to some aspects, the stable aqueous pharmaceutical composition comprises polysorbate 80 (PS80) at a concentration (% w/v) of about: 0.036%, 0.037%, 0.038%, 0.039%, 0.040%, 0.041%, 0.042%, 0.043% 0.044%, 0.045%, 0.046%, 0.047%, 0.048%, 0.049%, 0.050%, 0.051%, 0.052%, 0.053%, 0.054%, 0.055%, 0.056%, 0.057%, 0.058% 0.059%, 0.060%, 0.061%, 0.062%, 0.063%, 0.064%, 0.065%, 0.066%, 0.067%, 0.068% 0.069%, 0.070%, 0.071%, 0.072%, 0.073%, 0.074%, 0.075%, 0.080%, 0.081%, 0.082%, 0.083%, 0.084%, 0.085%, 0.086%, 0.087%, 0.088% 0.089%, 0.090%, 0.091%, 0.092%, 0.093%, 0.094%, or 0.095%. In some embodiments, the stable aqueous pharmaceutical composition comprises about 0.06% (w/v) PS80.

According to some aspects, the stable aqueous pharmaceutical composition comprises methionine at a concentration of about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL. In one embodiment, the methionine has a concentration of about 1.0 mg/mL.

According to some aspects, the stable aqueous pharmaceutical composition comprises EDTA at a concentration of about: 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, or 30 μg/mL. In one embodiment, the EDTA has a concentration of about 20 μg/mL.

Storage Time

In some embodiments, the DP stability is determined following storage for a specified period of time. In some embodiments, DP is stored for about 3 months or more, about 6 months or more, about 12 months or more, about 1.5 years or more, about 2 years or more, about 2.5 years or more, about 3 years or more, about 3.5 years or more, about 4 years or more, about 4.5 years or more, about 5 years or more, about 6 years or more, about 7 years or more, about 8 years or more, about 9 years or more, or about 10 years or more. In some embodiments, DP is stored for about 12 months or more, about 1.5 years or more, about 2 years or more, about 2.5 years or more, or about 3 years or more. In some embodiments, DP is stored for about 2 years or more.

Temperature

In some embodiments, the DP is stable following storage at a specific temperature for a specified period of time. In some embodiments, the temperature ranges between about: −10 to 50° C., 0 to 25° C., 1 to 20° C., 1 to 15° C., 2 to 10° C., or 2 to 5° C. In some embodiments, the temperature ranges between about: 2 to 8° C. In some embodiments, the temperature is about: −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. or 50° C.

In some embodiments, the DP is stable following storage for about 12 months or more, or for about 2 years or more and at a temperature ranging from about 2° C. to about 8° C. In some embodiments, the DP is stable following storage for about 12 months or more or for about 2 years or more and at a temperature of about 5° C. In some embodiments, the DP is stable following storage for about 12 months or more and at a temperature of about 25° C.

The stability of the presently disclosed aqueous pharmaceutical compositions, also referred to as drug product (DP), is determined based on specific amount or proportion of the bispecific EGFR-cMet antibody and other constituents of the DP as provided herein (such as, but not limited to, histidine and/or pharmaceutically acceptable histidine salts, sucrose, PS80, methionine, and EDTA), as well as the assessment of various factors. These factors include but are not limited to the color of the solution, the pH, the turbidity, number of subvisible particles, percentage of aglycosylated heavy chain (AGHC), percentage of new peak(s), percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, and/or percentage of PS80.

Stable DP as disclosed herein should not be construed to require all the factors listed herein but rather at least one, at least two, or at least three or more of those factors. In some embodiments, the stable disclosed DP exhibits the following results for at least one, at least two, at least three or more of the factors listed in detail below herein. In some embodiments, the stable DP exhibits the following results for all the factors listed in detail below herein.

Color of Solution

The Color of a DP solution is monitored and can be assessed to verify that the appearance of the solution is consistent with previous batches at release and over the shelf life. The color of the DP solution can reflect stability. In one embodiment, the stability of the DP is defined when having a color of solution spanning from colorless to about BY2 or less, to about BY4 or less, to about B2 or less, to about B4 or less, to about Y2 or less or to about Y4 or less as described in the European Pharmacopoeia 2.2.2, Degree of Coloration of Liquids European Pharmacopoeia (Ph. Eur.) 10th Edition monograph number 20202, July 2019.

In one embodiment, the stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

Measuring the pH of the DP solution allows confirmation that it is consistent with previous DP batches at release and over the shelf life. In one embodiment, the stability of the DP is defined when its pH is about: 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In one embodiment, the pH of the DP is about 5.7 after storage for about 12 months or more and at a temperature of about 5° C., after storage for about 12 months or more and at a temperature of about 25° C., and/or after storage for about 2 years or more and at a temperature of about 5° C. In one embodiment, the pH ranges from about 5.0 to about 6.4 after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when its pH ranges from about 5.2 to about 6.2 after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the DP is defined when its pH ranges from about 5.4 to about 6.0 after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the DP is defined when its pH ranges from about 5.3 to about 6.1 after storage for about 12 months or more and at a temperature of about 5° C., after storage for about 12 months or more and at a temperature of about 25° C., and/or after storage for about 2 years or more and at a temperature of about 5° C.

Turbidity

Turbidity allows measuring the presence of particles in the DP solution in order to ensure consistency with previous DP batches and applicable compendia guidance at release and over the shelf life. In one embodiment, the stability of the DP is defined when its turbidity value is about: 0, 1, 2, 3, 4, 5, 6, 7, 8, 9, 10, 11, 12, 13, 14, 15, 16, 17, 18, 19, or 20 nephelometric turbidity units (NTU) after storage for about 12 months or more and at a temperature of about 5° C., after storage for about 12 months or more and at a temperature of about 25° C., and/or after storage for about 2 years or more and at a temperature of about 5° C. In one embodiment, the stability of the DP is defined when its turbidity value is about or less than less 18 NTU after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when its turbidity value is about or less than less 13 NTU after storage for about 12 months or more and at a temperature of about 5° C., after storage for about 12 months or more and at a temperature of about 25° C., and/or after storage for about 2 years or more and at a temperature of about 5° C. In the most preferred embodiment, the stability of the DP is defined when its turbidity value is about or less than less 8 NTU after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the DP is defined when its turbidity value is about or less than less 6 NTU after storage for about 12 months or more and at a temperature of about 5° C., after storage for about 12 months or more and at a temperature of about 25° C., and/or after storage for about 2 years or more and at a temperature of about 5° C.

Particle Analysis

The stability of the DP is set to a specific threshold of particles contamination based on the average number of sub-visible particles. In one embodiment, the average number of particles present in the DP units tested should not exceed 100, 200, 300, 400, 500, 600, 700, 800, 900, 1000, 2000, 3000, 4000, 5000, or 6000, per container for particle size equal to 10 μm or greater. In one embodiment, the average number of particles present in the DP units tested should not exceed 6000 per container for particle size equal to 10 μm or greater. In one embodiment, the average number of particles present in the DP units tested should not exceed 100, 200, 300, 400, 500, or 600, per container for particle size equal to 25 μm or greater. In one embodiment, the average number of particles present in the DP units tested should not exceed 600 per container for particle size equal to 25 μm or greater.

cSDS Conditions

Capillary SDS-PAGE (cSDS)-reduced, like gel-based SDS-PAGE, is a method for separating denatured proteins based on molecular weight. This process allows quantifying DP purity and monitoring its stability at release and over the shelf life.

In one embodiment, the DP stability is defined based upon various results of cSDS variables (e.g. percent purity, aglycosylated heavy chain (AGHC), or presence of new peak) where the cSDS was performed under reduced or non-reduced conditions after storage for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a percent purity about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range there in between.

In one embodiment, the DP stability is defined as having an AGHC of about: 0.1%, 0.5%, 1%, 2%, 3%, 4%, 5%, 6%, 7%, 8%, 9%, 10%, 11%, 12%, 13%, 14%, 15% or any range there in between.

In one embodiment, the DP stability is defined as showing no new peak in the cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material.

In one embodiment, the DP stability is defined with a percent purity of about 88% or more, an AGHC of about 11% or more, and no new peak of more than 1.5% as compared to a reference material. In a preferred embodiment, the DP stability is defined with a percent purity of about 91% or more, with an AGHC of about 8% or less, and with no new peak of more than 1.0% compared to a reference material. In a most preferred embodiment, the DP stability is defined as having a percent purity of about 94% or more, AGHC of about 5% or less, and with no new peak of more than 1.0% as compared to a reference material. In a most preferred embodiment, the DP stability is defined as having a percent purity of about 93% or more, AGHC of about 6% or less, and with no new peak of more than 1.0% as compared to a reference material.

Capillary SDS-PAGE (cSDS), non-reduced like gel-based SDS-PAGE, is a method for separating proteins based on molecular weight. This process allows quantifying DP purity and monitoring its stability at release and over the shelf life.

In one embodiment, the DP stability is defined based upon various results of cSDS variables (e.g. percent purity or presence of new peak) where the cSDS was performed under non-reduced conditions after storage for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a percent purity about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range there in between.

In one embodiment, the DP stability is defined as showing no new peak in the cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material.

In one embodiment, the DP stability is defined with a percent purity of about 90% or more and no new peak of more than 1.5% as compared to a reference material. In a preferred embodiment, the DP stability is defined with a percent purity of about 94% or more, and with no new peak of more than 1.2% compared to a reference material. In a most preferred embodiment, the DP stability is defined as having a percent purity of about 97% or more and with no new peak of more than 1.0% as compared to a reference material.

Size-Exclusion HPLC (SE-HPLC) Results Consistent with Stability

SE-HPLC procedure allows assessing purity of the DP and monitoring its stability under non-denaturing conditions at release and over the shelf life.

In one embodiment, the DP stability is defined based upon various results of SE-HPLC variables such as the Main Component (MC), High Molecular Weight Species (HMWS), or Low Molecular Weight Species (LMWS)), after storage of the DP for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a MC of about: 80%, 81%, 82%, 83%, 84%, 85%, 86%, 87%, 88%, 89%, 90%, 91%, 92%, 93%, 94%, 95%, 96%, 97%, 98%, 99% or equal to about 100% or any range there in between. In one embodiment, the DP stability is defined as having a MC of about 90% or more. In one embodiment, the DP stability is defined as having a MC of about 92% or more. In a preferred embodiment, the DP stability is defined as having a MC of about 95% or more. In the most preferred embodiment, the DP stability is defined as having a MC of about 97% or more. In a most preferred embodiment, the DP stability is defined as having a MC of about 98% or more.

In one embodiment, the DP stability is defined as having a HMWS of about: 0.1%, 0.5%, 1%, 2%, 3%, 4%, 5%, 6%, 7%, 8%, 9%, 10%, 11%, 12%, 13%, 14%, 15% or any range there in between. In one embodiment, the DP stability is defined as having a HMWS of about 10% or less. In one embodiment, the DP stability is defined as having a HMWS of about 8% or less. In a preferred embodiment, the DP stability is defined as having a HMWS of about 5% or less. In the most preferred embodiment, the DP stability is defined as having a HMWS of about to 3% or less. In a preferred embodiment, the DP stability is defined as having a HMWS of about 2% or less.

In one embodiment, the DP stability is defined as having a LMWS of about: 0.1%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%. In one embodiment, the DP stability is defined as having a LMWS of about 5% or less. In a preferred embodiment, the DP stability is defined as having a LMWS of about 2% or less. In a most preferred embodiment, the DP stability is defined as having a LMWS of about 1% or less.

Capillary Isoelectric Focusing (cIEF)

The cIEF, like isoelectric gel electrophoresis (IEF) methods, separates proteins on the basis of overall charge or isoelectric point (pI). This procedure allows monitoring the distribution of charge-based isoforms of the drug product at release and over the shelf life. In one embodiment, the DP stability is defined based upon various results of cIEF variables such as the Main Peak (MP), the sum of acidic peaks or the sum of basic peaks, after DP storage for about 12 months or more and at a temperature of about 25° C., and/or after storage for about 2 years or more and at a temperature of about 5° C.

In one embodiment, the DP stability is defined as having a cIEF with a MP of about: 20%, 25%, 30%, 35%, 40%, 45%, 50%, 55%, 60%, 65%, 70%, 75%, 80%, 85%, 90%, 95%, or 100% or any range there in between after DP storage for about 12 months or more and at a temperature of about 25° C., and/or after storage for about 2 years or more and at a temperature of about 5° C. In one embodiment, the DP stability is defined as having a cIEF with a MP ranging from about 30% to about 90% after DP storage for about 12 months or more and at a temperature of about 25° C., and/or after storage for about 2 years or more and at a temperature of about 5° C. In one embodiment, the DP stability is defined as having a cIEF with a MP ranging from about 37% to about 87% after DP storage for about 12 months or more and at a temperature of about 25° C., and/or after storage for about 2 years or more and at a temperature of about 5° C. In a preferred embodiment, the DP stability is defined as having a cIEF with a MP ranging from about 47% to about 87% after DP storage for about 12 months or more and at a temperature of about 25° C., and/or after storage for about 2 years or more and at a temperature of about 5° C. In a preferred embodiment, the DP stability is defined as having a cIEF with a MP ranging from about 46% to about 87% after DP storage for about 12 months or more and at a temperature of about 25° C., and/or after storage for about 2 years or more and at a temperature of about 5° C. In the most preferred embodiment, the DP stability is defined as having a cIEF with a MP ranging from about 57% to about 87% after DP storage for about 12 months or more and at a temperature of about 25° C., and/or after storage for about 2 years or more and at a temperature of about 5° C. In a most preferred embodiment, the DP stability is defined as having a cIEF with a MP ranging from about 66% to about 83% after DP storage for about 12 months or more and at a temperature of about 25° C., and/or after storage for about 2 years or more and at a temperature of about 5° C.

In one embodiment, the DP stability is defined as having a cIEF with a with a sum of acidic peaks totaling to about: 1%, 5%, 10%, 15%, 20%, 25%, 30%, 35%, 40%, 45%, 50%, 55%, 60%, 65%, 70%, 75% or 80% or any range there in between after DP storage for about 12 months or more and at a temperature of about 25° C., and/or after storage for about 2 years or more and at a temperature of about 5° C. In one embodiment, the DP stability is defined as having a cIEF with a sum of acidic peaks totaling to about 5% to about 65% after DP storage for about 12 months or more and at a temperature of about 25° C., and/or after storage for about 2 years or more and at a temperature of about 5° C. In one embodiment, the DP stability is defined as having a cIEF with a sum of acidic peaks totaling to about 10% to about 60% after DP storage for about 12 months or more and at a temperature of about 25° C., and/or after storage for about 2 years or more and at a temperature of about 5° C. In one embodiment, the DP stability is defined as having a cIEF with a sum of acidic peaks totaling to about 10% to about 50% after DP storage for about 12 months or more and at a temperature of about 25° C., and/or after storage for about 2 years or more and at a temperature of about 5° C. In a preferred embodiment, the DP stability is defined as having a cIEF with a sum of acidic peaks totaling to about 10 to about 50% after DP storage for about 12 months or more and at a temperature of about 25° C., and/or after storage for about 2 years or more and at a temperature of about 5° C. In a preferred embodiment, the DP stability is defined as having a cIEF with a sum of acidic peaks totaling to about 10 to about 40% after DP storage for about 12 months or more and at a temperature of about 25° C., and/or after storage for about 2 years or more and at a temperature of about 5° C. In a most preferred embodiment, the DP stability is defined as having a cIEF with a sum of acidic peaks totaling to about 10% to about 40% after DP storage for about 12 months or more and at a temperature of about 25° C., and/or after storage for about 2 years or more and at a temperature of about 5° C. In a most preferred embodiment, the DP stability is defined as having a cIEF with a sum of acidic peaks totaling to about 15% to about 31% after DP storage for about 12 months or more and at a temperature of about 25° C., and/or after storage for about 2 years or more and at a temperature of about 5° C.

In one embodiment, the DP stability is defined as having a cIEF with a sum of basic peaks totaling about: 1%, 2%, 3%, 4%, 5%, 6%, 7%, 8%, 9%, 10%, 11%, 12%, 13%, 14%, 15%, 16%, 17%, 18%, 19% or 20% or any range there in between after DP storage for about 12 months or more and at a temperature of about 25° C., and/or after storage for about 2 years or more and at a temperature of about 5° C. In one embodiment, the DP stability is defined as having a cIEF with a sum of basic peaks totaling about 12% or less after DP storage for about 12 months or more and at a temperature of about 25° C., and/or after storage for about 2 years or more and at a temperature of about 5° C. In one embodiment, the DP stability is defined as having a cIEF with a sum of basic peaks totaling about 10% or less after DP storage for about 12 months or more and at a temperature of about 25° C., and/or after storage for about 2 years or more and at a temperature of about 5° C. In a preferred embodiment, the DP stability is defined as having a cIEF with a sum of basic peaks totaling less than or about 10% after DP storage for about 12 months or more and at a temperature of about 25° C., and/or after storage for about 2 years or more and at a temperature of about 5° C. In a preferred embodiment, the DP stability is defined as having a cIEF with a sum of basic peaks totaling less than or about 8% after DP storage for about 12 months or more and at a temperature of about 25° C., and/or after storage for about 2 years or more and at a temperature of about 5° C. In a most preferred embodiment, the DP stability is defined as having a cIEF with a sum of basic peaks totaling less than or about 8% after DP storage for about 12 months or more and at a temperature of about 25° C., and/or after storage for about 2 years or more and at a temperature of about 5° C. In a most preferred embodiment, the DP stability is defined as having a cIEF with a sum of basic peaks totaling less than or about 5% after DP storage for about 12 months or more and at a temperature of about 25° C., and/or after storage for about 2 years or more and at a temperature of about 5° C.

Protein Concentration

Protein concentration of the DP allows verifying that it is consistent with previous DP batches at release and over the shelf life. Quantification of protein concentration can be accomplished by measuring the UV light absorbance of the drug product solution at 280 nm (A280).

In one embodiment, the DP stability is defined as having a protein concentration of about: 10 mg/mL, 20 mg/mL, 30 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, or 65 mg/mL after DP storage for about 12 months or more and at a temperature of about 25° C., and/or after storage for about 2 years or more and at a temperature of about 5° C. In one embodiment, the DP stability is defined as having a protein concentration of about 40 mg/mL to about 60 mg/mL after DP storage for about 12 months or more and at a temperature of about 25° C., and/or after storage for about 2 years or more and at a temperature of about 5° C. In a preferred embodiment, the DP stability is defined as having a protein concentration of about 45 mg/mL to about 55 mg/mL after DP storage for about 12 months or more and at a temperature of about 25° C., and/or after storage for about 2 years or more and at a temperature of about 5° C. In a preferred embodiment, the DP stability is defined as having a protein concentration of about 43 mg/mL to about 57 mg/mL after DP storage for about 12 months or more and at a temperature of about 25° C., and/or after storage for about 2 years or more and at a temperature of about 5° C. In a most preferred embodiment, the DP stability is defined as having a protein concentration of about 47 mg/mL to 54 mg/mL after DP storage for about 12 months or more and at a temperature of about 25° C., and/or after storage for about 2 years or more and at a temperature of about 5° C. In a most preferred embodiment, the DP stability is defined as having a protein concentration of about 45 mg/mL to 55 mg/mL after DP storage for about 12 months or more and at a temperature of about 25° C., and/or after storage for about 2 years or more and at a temperature of about 5° C.

Peptide Mapping

Post-translational modifications (PTMs), such as oxidation, deamidation, and isomerization, are enzymatic modifications that may be detected in the structure of an antibody. In some embodiments, the PD stability is assessed based on level of PTMs in the antibody. Test articles are enzymatically digested to yield peptide segments. These peptides are then evaluated by for instance by mass spectrometry (MS), by tandem mass spectrometry (MS-MS) or Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Drug Product Potency

In vitro binding of the DP to EGFR and/or c-Met allows assessing the level of DP stability. This binding can be assessed by using, but not limited to, a homogeneous competitive time resolved fluorescence resonance energy transfer (TR-FRET) assay.

EGFR Binding Activity

In one embodiment, the DP stability is defined as having an EGFR binding activity, relative to a reference, of about: 20%, 30%, 40%, 50%, 60%, 70%, 80%, 90%, 100%, 110%, 120%, 130%, 140%, 150%, 160%, 170%, 180%, 190%, or 200% or any range there in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined as having an EGFR binding activity ranging from about 50% to about 150% relative to a reference after DP storage for about 12 months or more and at a temperature of about 25° C., and/or after storage for about 2 years or more and at a temperature of about 5° C. In one embodiment, the DP stability is defined as having an EGFR binding activity ranging from about 60% to about 140% relative to a reference after DP storage for about 12 months or more and at a temperature of about 25° C., and/or after storage for about 2 years or more and at a temperature of about 5° C. In a preferred embodiment, the DP stability is defined as having an EGFR binding activity ranging from about 60% to about 140% relative to a reference after DP storage for about 12 months or more and at a temperature of about 25° C., and/or after storage for about 2 years or more and at a temperature of about 5° C. In a preferred embodiment, the DP stability is defined as having an EGFR binding activity ranging from about 65% to about 130% relative to a reference after DP storage for about 12 months or more and at a temperature of about 25° C., and/or after storage for about 2 years or more and at a temperature of about 5° C. In a most preferred embodiment, the DP stability is defined as having an EGFR binding activity ranging from about 80% to about 120% relative to a reference after DP storage for about 12 months or more and at a temperature of about 25° C., and/or after storage for about 2 years or more and at a temperature of about 5° C. In a most preferred embodiment, the DP stability is defined as having an EGFR binding activity ranging from about 70% to about 130% relative to a reference after DP storage for about 12 months or more and at a temperature of about 25° C., and/or after storage for about 2 years or more and at a temperature of about 5° C.

cMet Binding Activity Results Consistent with Stability

In one embodiment, the DP stability is defined as having a cMet binding activity, relative to a reference, of about: 20%, 30%, 40%, 50%, 60%, 70%, 80%, 90%, 100%, 110%, 120%, 130%, 140%, 150%, 160%, 170%, 180%, 190%, or 200% or any range there in between after DP storage for about 12 months or more and at a temperature of about 25° C., and/or after storage for about 2 years or more and at a temperature of about 5° C. In one embodiment, the DP stability is defined as having a cMet binding activity ranging from about 50% to about 150% relative to a reference after DP storage for about 12 months or more and at a temperature of about 25° C., and/or after storage for about 2 years or more and at a temperature of about 5° C. In one embodiment, the DP stability is defined as having a cMet binding activity ranging from about 60% to about 140% relative to a reference after DP storage for about 12 months or more and at a temperature of about 25° C., and/or after storage for about 2 years or more and at a temperature of about 5° C. In a preferred embodiment, the DP stability is defined as having a cMet binding activity ranging from about 60% to about 140% relative to a reference after DP storage for about 12 months or more and at a temperature of about 25° C., and/or after storage for about 2 years or more and at a temperature of about 5° C. In a preferred embodiment, the DP stability is defined as having a cMet binding activity ranging from about 65% to about 125% relative to a reference after DP storage for about 12 months or more and at a temperature of about 25° C., and/or after storage for about 2 years or more and at a temperature of about 5° C. In a most preferred embodiment, the DP stability is defined as having a cMet binding activity ranging from about 80% to about 120% relative to a reference after DP storage for about 12 months or more and at a temperature of about 25° C., and/or after storage for about 2 years or more and at a temperature of about 5° C. In a most preferred embodiment, the DP stability is defined as having a cMet binding activity ranging from about 76% to about 125% relative to a reference after DP storage for about 12 months or more and at a temperature of about 25° C., and/or after storage for about 2 years or more and at a temperature of about 5° C.

Polysorbate 80 (PS80)

In one embodiment, the DP stability is defined by a PS80 concentration in percentage weight to volume of about: 0.005%, 0.01%, 0.02%, 0.03%, 0.04%, 0.05%, 0.06%, 0.08%, 0.09%, 0.1%, 0.11%, 0.12%, 0.13%, 0.14%, or 0.15% or any range there in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined by a PS80 concentration of about 0.02% to about 0.1% after DP storage for about 12 months or more and at a temperature of about 25° C., and/or after storage for about 2 years or more and at a temperature of about 5° C. In one embodiment, the DP stability is defined by a PS80 concentration of about 0.03% to about 0.09%. In a preferred embodiment, the DP stability is defined by a PS80 concentration of about 0.04% to about 0.08% after DP storage for about 12 months or more and at a temperature of about 25° C., and/or after storage for about 2 years or more and at a temperature of about 5° C. In one embodiment, the DP stability is defined by a PS80 concentration of about 0.02% to about 0.09%. In a preferred embodiment, the DP stability is defined by a PS80 concentration of about 0.03% to about 0.08% after DP storage for about 12 months or more and at a temperature of about 25° C., and/or after storage for about 2 years or more and at a temperature of about 5° C. In a most preferred embodiment, the DP stability is defined with a PS80 concentration of about 0.05% to about 0.08% after DP storage for about 12 months or more and at a temperature of about 25° C., and/or after storage for about 2 years or more and at a temperature of about 5° C. In a most preferred embodiment, the DP stability is defined with a PS80 concentration of about 0.04% to about 0.08% after DP storage for about 12 months or more and at a temperature of about 25° C., and/or after storage for about 2 years or more and at a temperature of about 5° C.

In one embodiment, the total volume of the stable aqueous pharmaceutical composition (or DP) ranges from about 5 mL to about 10 mL. In one embodiment, the total volume of the stable aqueous pharmaceutical composition (or DP) ranges from about 0.5 mL to about 20 mL, from about 1 mL to about 15 mL, from about 5 mL to about 10 mL, or from about 6 mL to about 8 mL. In one embodiment, the total volume of the stable aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any ranges there in between.

Methods

Provided herein is a method of treating cancer in a subject in need thereof. In some embodiments, the cancer is a lung cancer. In some embodiments, the lung cancer is a non-small cell lung cancer (NSCLC). The method comprises administering to the subject a stable aqueous pharmaceutical composition as disclosed herein. In one embodiment, the administration is intravenous.

Also provided herein is a method for preparing a stable aqueous pharmaceutical composition of a bispecific antibody targeting EGFR and cMet. The bispecific antibody targeting EGFR and cMet used in this method comprises a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively (see table 14). The method also comprises combining a composition comprising about 50 mg/mL of the bispecific antibody, about 10 mM histidine and/or pharmaceutically acceptable histidine salt, about 8.5% Sucrose, and about 1 mg/mL L-methionine with polysorbate 80 to a final concentration of about 0.06% (w/v) and EDTA to a final concentration of about 20 μg/mL, wherein the stable aqueous pharmaceutical composition has about pH 5.7.

In one embodiment, the first heavy chain (HC1) of the bispecific EGFR-cMet antibody comprises a HC1 constant domain 3 (HC1 CH3) and a HC1 variable region 1 (VH1). In another embodiment, the second heavy chain (HC2) of the bispecific EGFR-cMet antibody comprises a HC2 constant domain 3 (HC2 CH3) and a HC2 variable region 2 (VH2).

In one embodiment, the bispecific EGFR-cMet antibody comprises an HC1 variable region comprising the amino acid sequence of SEQ ID NO:13 and a LC1 variable region comprising the amino acid sequence of SEQ ID NO:14 (see table 14).

In one embodiment, the bispecific EGFR-cMet antibody comprises a HC2 variable region comprising the amino acid sequence of SEQ ID NO:15 and a LC2 variable region comprising the amino acid sequence of SEQ ID NO:16 (see table 14).

In one embodiment, the HC1 comprises the amino acid sequence of SEQ ID NO:17 and the HC2 comprises the amino acid sequence of SEQ ID NO:19 (see table 14).

In another embodiment, the LC1 comprises the amino acid sequence of SEQ ID NO:18 and the LC2 comprises the amino acid sequence of SEQ ID NO:20 (see table 14).

In one embodiment, the method further comprises filtering the stable aqueous pharmaceutical composition. In another embodiment, the filtering is performed with one or more 0.22 μm sterilizing filters. In one embodiment, sterilizing filter has a pore size of about: 0.10 μm, 0.15 μm, 0.20 μm, 0.25 μm, 0.30 μm, 0.35 μm, 0.40 μm, 0.45 μm, 0.50 μm, 0.55 μm, 0.60 μm, 0.65 μm, 0.70 μm, or 0.75 μm.

The stable aqueous pharmaceutical compositions disclosed herein can be packaged into kits, containers, packs, dispensers, or vials.

Provided herein is a kit comprising the disclosed stable aqueous pharmaceutical and instructions for use thereof.

Also provided herein is an article of manufacture comprising a container holding the disclosed stable aqueous pharmaceutical composition. In some embodiments, the container is a vial with a stopper pierceable by a syringe.

### Illustrative Embodiments

Provided here are illustrative embodiments of the disclosed technology. These embodiments are illustrative only and do not limit the scope of the present disclosure or of the claims attached hereto.

Embodiment 1. A stable aqueous pharmaceutical composition comprising:

a) about 44 mg/mL to about 56 mg/mL of a bispecific epidermal growth factor receptor (EGFR)/hepatocyte growth factor receptor (c-Met) antibody, the bispecific antibody comprising:
a first heavy chain (HC1) comprising a HC1 variable region 1 (VH1);
a first light chain (LC1) comprising a light chain variable region 1 (VL1);
a second heavy chain (HC2) comprising a HC2 variable region 2 (VH2); and
a second light chain (LC2) comprising a light chain variable region 2 (VL2),
wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively, the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively;
b) about 8 mM to about 12 mM of histidine and/or pharmaceutically acceptable histidine salt,
c) about 6.8% (w/v) to about 10.2% (w/v) of sucrose,
d) about 0.036% (w/v) to about 0.084% (w/v) of polysorbate 80 (PS80),
e) about to 0.8 mg/mL to about 1.2 mg/mL of methionine,
f) about 16 μg/mL to about 24 μg/mL of ethylenediaminetetraacetic acid (EDTA); and
g) a pH from about 5.2 to about 6.2.

Embodiment 2. The stable aqueous pharmaceutical composition of embodiment 1, wherein the bispecific EGFR-cMet antibody comprises an HC1 variable region comprising the amino acid sequence of SEQ ID NO:13 and a LC1 variable region comprising the amino acid sequence of SEQ ID NO:14.

Embodiment 3. The stable aqueous pharmaceutical composition of embodiment 1 or embodiment 2, wherein the bispecific EGFR-cMet antibody comprises a HC2 variable region comprising the amino acid sequence of SEQ ID NO:15 and a LC2 variable region comprising the amino acid sequence of SEQ ID NO:16.

Embodiment 4. The stable aqueous pharmaceutical composition of any one of embodiments 1-3, wherein the HC1 comprises the amino acid sequence of SEQ ID NO:17 and the LC1 comprises the amino acid sequence of SEQ ID NO:18.

Embodiment 5. The stable aqueous pharmaceutical composition of any one of embodiments 1-4, wherein the HC2 comprises the amino acid sequence of SEQ ID NO:19 and the LC2 comprises the amino acid sequence of SEQ ID NO:20.

Embodiment 6. The stable aqueous pharmaceutical composition of any one of embodiments 1-5, wherein the bispecific EGFR-cMet antibody is amivantamab.

Embodiment 7. The stable aqueous pharmaceutical composition of any one of embodiments 1-6, wherein the bispecific EGFR-cMet antibody has a concentration of about 50 mg/mL.

Embodiment 8. The stable aqueous pharmaceutical composition of any one of embodiments 1-7, wherein the histidine and/or pharmaceutically acceptable histidine salt has a concentration of about 10 mM.

Embodiment 9. The stable aqueous pharmaceutical composition of any one of embodiments 1-8, wherein the histidine and/or pharmaceutically acceptable histidine salt comprises L-histidine and L-histidine hydrochloride monohydrate.

Embodiment 10. The stable aqueous pharmaceutical composition of any one of embodiments 1-9, comprising about 8.5% (w/v) sucrose.

Embodiment 11. The stable aqueous pharmaceutical composition of any one of embodiments 1-10, comprising about 0.06% (w/v) PS80.

Embodiment 12. The stable aqueous pharmaceutical composition of any one of embodiments 1-11, wherein the methionine has a concentration of about 1 mg/mL.

Embodiment 13. The stable aqueous pharmaceutical composition of any one of embodiments 1-12, wherein the EDTA has a concentration of about 20 μg/mL.

Embodiment 14. The stable aqueous pharmaceutical composition of any one of embodiments 1-13, wherein the pH is about 5.7.

Embodiment 15. The stable aqueous pharmaceutical composition of any one of embodiments 1-14, wherein stability is defined based on color of solution, pH, turbidity, number of subvisible particles, percentage of aglycosylated heavy chain (AGHC), percentage of new peak(s), percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, percentage of PS80, or any combination thereof.

Embodiment 15a. The stable aqueous pharmaceutical composition of any one of claims 1-14, wherein stability is defined based on color of solution, pH, turbidity, number of subvisible particles, percentage of purity, percentage of aglycosylated heavy chain (AGHC), percentage of new peak(s) as measured by cSDS-reduced, the percentage of purity and new peak(s) as measured by cSDS-non-reduced, percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, percentage of PS80, or any combination thereof.

Embodiment 15b. The stable aqueous pharmaceutical composition of any one of claims 1-15, wherein the stable aqueous pharmaceutical composition is stable at a temperature of about 2-8° C. for at least two years.

Embodiment 16. The stable aqueous pharmaceutical composition of any one of embodiments 1-15, wherein the total volume of the composition ranges from about 5 mL to about 10 mL.

Embodiment 16a. The stable aqueous pharmaceutical composition of any one of embodiments 1-14, comprising 50 mg/mL of the bispecific EGFR-cMet antibody, 10 mM of the histidine and/or pharmaceutically acceptable histidine salt, 8.5% (w/v) sucrose, 0.06% (w/v) PS80, 1 mg/mL of the methionine, and 20 μg/mL of the EDTA.

Embodiment 17. A method of treating cancer in a subject in need thereof, the method comprising administering to the subject the pharmaceutical composition of any one of embodiments 1-16a.

Embodiment 18. The method of embodiment 17, wherein the administering is intravenous.

Embodiment 19. A method for preparing a stable aqueous pharmaceutical composition of a bispecific antibody targeting EGFR and cMet, the bispecific antibody targeting EGFR and cMet comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively; the method comprising:

combining a composition comprising about 50 mg/mL of the bispecific antibody, about 10 mM histidine and/or pharmaceutically acceptable histidine salt, about 8.5% Sucrose, and about 1 mg/mL L-methionine with polysorbate 80 to a final concentration of about 0.06% (w/v) and EDTA to a final concentration of about 20 μg/mL, wherein the stable aqueous pharmaceutical composition has about pH 5.7.

Embodiment 20. The method of embodiment 19, wherein the bispecific EGFR-cMet antibody comprises an HC1 variable region comprising the amino acid sequence of SEQ ID NO:13 and a LC1 variable region comprising the amino acid sequence of SEQ ID NO:14.

Embodiment 21. The method of any one of embodiments 19-21, wherein the bispecific EGFR-cMet antibody comprises a HC2 variable region comprising the amino acid sequence of SEQ ID NO:15 and a LC2 variable region comprising the amino acid sequence of SEQ ID NO:16.

Embodiment 22. The method of any one of embodiments 19-21, wherein the antibody comprises a heavy chain 1 (HC1) comprising the amino acid sequence of SEQ ID NO:17 and a light chain 1 (LC1) comprising the amino acid sequence of SEQ ID NO:18.

Embodiment 23. The method of any one of embodiments 19-22, wherein the antibody comprises a HC2 comprising the amino acid sequence of SEQ ID NO:19 and a LC2 comprising the amino acid sequence of SEQ ID NO:20.

Embodiment 24. The method of any one of embodiments 19-23, wherein the antibody is amivantamab.

Embodiment 25. A kit comprising the stable aqueous pharmaceutical composition of any one of embodiments 1-16 and instructions for use thereof.

Embodiment 26. An article of manufacture comprising a container holding a stable aqueous pharmaceutical composition in accordance with any one of embodiments 1-16.

Embodiment 27. The article of manufacture according to embodiment 26, wherein the container is a vial with a stopper pierceable by a syringe.

Embodiment 28. A pharmaceutical composition of any one of embodiments 1-16 for use in the treatment of cancer.

Embodiment 29. A pharmaceutical composition of any one of embodiments 1-16 for use in the preparation of a medicine.

Embodiment 30. Use of a pharmaceutical composition for treating cancer in a subject in need thereof by administering the pharmaceutical composition of any one of embodiments 1-16.

Embodiment 31. Use of a pharmaceutical composition according to embodiment 30, wherein the administration is intravenous.

### EXAMPLES

The following examples are provided to further describe some of the embodiments disclosed herein. The examples are intended to illustrate, not to limit, the disclosed embodiments.

Description of Analytical Tests Used Herein

Analytical Tests—General Characterization

Color of Solution

Color of solution is monitored for drug product to assess appearance and ensure it is consistent with previous batches at release and over the shelf life. Color of solution may be an indicator of product stability. To determine Color of solution, test samples are visually compared to a defined set of reference solutions.

A defined volume of liquid content is transferred into a pre-scored ampoule of same dimensions as the reference solutions. Then the content of the ampoule is visually compared to European Pharmacopoeia color reference solutions. The degree of color is determined in diffuse daylight, viewed against a white background.

Color of Solution Material and Methods

Materials and methods are as described in European Pharmacopoeia 2.2.2, Degree of Coloration of Liquids European Pharmacopoeia (Ph. Eur.) 10th Edition monograph number 20202, July 2019. Briefly, test articles are compared against B (Brown), BY (Brownish-Yellow), and Y (Yellow) Color Reference Solution Sets.

Color of Solution results consistent with stability. In one embodiment, stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

pH Materials and Methods—A daily calibrated electronic pH meter with standardized pH electrode is used to measure the pH of test articles. All calibration solutions, reference buffers, and test articles are equilibrated to, and maintained at, 25° C. prior to and during testing.

pH results consistent with stability. In one embodiment, stability is defined as having a pH range of 5.0 to 6.4 after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a pH range of 5.2 to 6.2 after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a pH range of 5.4 to 6.0 after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a pH range of 5.3 to 6.1 after storage for about 12 months or more and at a temperature of about 5° C., after storage for about 12 months or more and at a temperature of about 25° C., and/or after storage for about 2 years or more and at a temperature of about 5° C.

Turbidity

Turbidity Materials and Methods—The materials and methods are based on European Pharmacopoeia 2.2.1, Clarity and Degree of Opalescence of Liquids.

Turbidity results consistent with stability. Test results are reported in nephelometric turbidity units (NTU). In one embodiment, stability is defined as having a turbidity value of about 18 NTU or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a turbidity value of about 13 NTU or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a turbidity value of about 8 NTU or less after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a turbidity value of about 6 NTU or less after storage for about 12 months or more and at a temperature of about 5° C., after storage for about 12 months or more and at a temperature of about 25° C., and/or after storage for about 2 years or more and at a temperature of about 5° C.

Analytical Tests—Particulate Matter

Particulate Matter (Sub-visible) Materials and Methods—All materials and methods are compliant with United States Pharmacopeia <788> Particulate Matter. A Compendial compliant Liquid Particle Counter instrument equipped with a compendial volume sampler set-up is used. Test articles are equilibrated to room temperature for at least 60 minutes, but no longer than 10 hours, prior to testing. Test article vials are pooled in manner compliant with United States Pharmacopeia <788> Particulate Matter. As instructed by United States Pharmacopeia <788> Particulate Matter, four portions of pooled test article, each of appropriate volume, are removed and the number of particles equal to or greater than 10 μm and 25 μm are counted per portion. Results obtained for the first portion are disregarded and the remaining three results are used to calculate the mean number of particles for the preparation examined.

Particle Analysis (sub-vis) compendia compliant results—Testing results are to comply with United States Pharmacopoeia <788> Particulate Matter, European Pharmacopoeia 2.9.19, and Japanese Pharmacopoeia XVII/6.07 Particulate Contamination: Sub-visible particles. As such, the average number of particles present in the units tested should not exceed 6000 particles per container for particles size equal to 10 μm or greater and should not exceed 600 particles per container for particles size equal to 25 μm or greater.

Analytical Tests—Purity

Capillary electrophoresis sodium dodecyl sulfate (cSDS)-Reduced

cSDS Reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer and certified protein molecular weight standards spanning a range of approximately 10 to 148 kDa. The instrument's ultraviolet absorption spectrophotometer detector is set at a wavelength of 220 nm and the capillary temperature is set to 25° C. For reducing sample treatment conditions, the test article (in duplicate) is mixed with SDS and 2-mercaptoethanol and then heated for a defined time and temperature to fully denature and reduce the protein. The reduced sample is injected electro-kinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected for the light chain, heavy chain, and aglycosylated heavy chain (AG HC).

cSDS Reduced results consistent with stability. In one embodiment, stability is defined as having a percent purity ≥88.0%, AG HC≤11.0%, and no new peak >1.5% compared to a validated stock of amivantamab Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a percent purity about or more than 91.0%, AG HC less than or about 8.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a percent purity about or more than 91.0%, AG HC less than or about 8.0%, and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity about or more than 94.0%, AG HC less than or about 5.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a percent purity about or more than 93.0%, AG HC less than or about 6.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

Capillary Electrophoresis Sodium Dodecyl Sulfate (cSDS)-Non-Reduced

cSDS Non-reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer, certified protein molecular weight standards spanning a range of approximately 10 to 148 kDa, and a validated amivantamab reference material sample. The instrument's ultraviolet absorption spectrophotometer detector is set at a wavelength of 220 nm and the capillary temperature is set to 25° C. For non-reduced sample treatment conditions, the test article (in duplicate) is mixed with SDS and the alkylating reagent (N-Ethylmaleimide, to prevent disulfide bond shuffling or reformation). It is then heated for a defined time and temperature to fully denature the protein and minimize formation of fragments and artefact bands. The non-reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected. The data is also analyzed for the presence of new peaks versus amivantamab reference material. Percent purity is defined as percent heavy chain+percent light chain.

cSDS Non-Reduced results consistent with stability. In one embodiment, stability is defined as having a percent purity of about 88.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a percent purity of about 90.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as percent purity of about 90.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as percent purity of about 94.0% or more and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity of about 94.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a percent purity of about 97.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

Size Exclusion High Performance Liquid Chromatography (SE-HPLC)

SE-HPLC Materials and Methods—Reference Material and test articles are diluted to a target protein concentration. A 20 μl volume of analyte is injected onto a 7.8 mm×30 cm size exclusion column with 5 μm particle size silica base, with a fractionation range of 10 to 500 kDa. Aqueous phosphate buffer is used as the mobile phase at a flow rate of 0.7 mL/minute and the absorbance of the eluate is monitored continuously at 280 nm. Monomer (main component or main peak), aggregates (high molecular weight species, or HMWS), and fragments (low molecular weight species, or LMWS) are separated on the column and elute at different retention times. The amounts of these species are measured by monitoring peak absorbance at 280 nm.

SE-HPLC Results Consistent with Stability

Main Component—In one embodiment, stability is defined as having a Main Component about 90.0% or more after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a Main Component about 92.0% or more after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Component about 95.0% or more after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Component about 97.0% after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a Main Component about 98.0% after storage for about 12 months or more and at a temperature of about 5° C., after storage for about 12 months or more and at a temperature of about 25° C., and/or after storage for about 2 years or more and at a temperature of about 5° C. High Molecular Weight Species (HMWS)—In one embodiment, stability is defined as having a HMWS of about 10.0% or less after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a HMWS of about 8.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a HMWS of about 5.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a HMWS of about 3.0% or less after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a HMWS of about 2.0% or less after storage for about 12 months or more and at a temperature of about 5° C., after storage for about 12 months or more and at a temperature of about 25° C., and/or after storage for about 2 years or more and at a temperature of about 5° C. Low Molecular Weight Species (LMWS)—In one embodiment, stability is defined as having a LMWS about 5.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a LMWS of about 2.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a LMWS about 1.0% or less after storage for about 12 months or more and at a temperature of about 5° C., after storage for about 12 months or more and at a temperature of about 25° C., and/or after storage for about 2 years or more and at a temperature of about 5° C.

Capillary Isoelectric Focusing (cIEF)

cIEF Materials and Methods—The analytical procedure is performed on a commercially available imaging cIEF analyzer equipped with an auto sampler. Analysis employs a 100-μm inner wall-coated silica capillary with an outer wall polyimide coating. In addition, an analyte solution of dilute phosphoric acid and methylcellulose, a catholyte solution of sodium hydroxide and methylcellulose, and defined type and amount of ampholytes are used. The test articles are treated with carboxypeptidase B (CPB) to remove C-terminal lysine and eliminate ambiguities introduced by the presence of multiple C-terminal variants for each charged species. The instrument's autosampler is set to 4° C. for both pre-focusing and focusing. The Pre-focusing voltage and time are 1500 V and 1 minute respectively. The Focusing voltage and time are 3000 V and 7 minutes respectively.

cIEF Results Consistent with Stability

Main Peak—In one embodiment, stability is defined as having a Main Peak of 37-87% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Peak of 47-87% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Peak of 46-87% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Peak of 57-87% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Peak of 66-83% after storage for about 12 months or more and at a temperature of about 5° C., after storage for about 12 months or more and at a temperature of about 25° C., and/or after storage for about 2 years or more and at a temperature of about 5° C.

Sum of acidic peaks—In one embodiment, stability is defined as having a Sum of acidic peaks totaling 10-60% after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a Sum of acidic peaks totaling 10-50% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of acidic peaks totaling 10-50% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of acidic peaks totaling 10-40% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of acidic peaks totaling 10-40% after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a Sum of acidic peaks totaling 15-31% after storage for about 12 months or more and at a temperature of about 5° C., after storage for about 12 months or more and at a temperature of about 25° C., and/or after storage for about 2 years or more and at a temperature of about 5° C.

Sum of basic peaks—In one embodiment, stability is defined as having a Sum of basic peaks totaling about 12.0% or less after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a Sum of basic peaks totaling about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of basic peaks totaling about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of basic peaks totaling about 8.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of basic peaks totaling about 8.0% or less after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as having a Sum of basic peaks totaling about 5.0% or less after storage for about 12 months or more and at a temperature of about 5° C., after storage for about 12 months or more and at a temperature of about 25° C., and/or after storage for about 2 years or more and at a temperature of about 5° C.

Analytical Tests—Quantity

Protein Concentration by A280

Protein concentration of the drug product is determined by quantification of the absorbance at 280 nm (A280).

Protein Concentration by A280 Materials and Methods

Measurement of protein concentration is performed using a qualified and calibrated double beam UV-Vis spectrophotometer. Test articles are diluted 1:125 using 0.9% (w/v) NaCl. Samples are measured using quartz semi-micro cuvettes (1.4 ml) with a 1 cm path length and black or frosted sides. The Spectrophotometer is set to a Wavelength of 280 nm, a slit width of 1 nm, and a response of one (1) second. 0.9% (w/v) NaCl is used as the Blank control. Protein concentration (mg/mL) is calculated by dividing the product of the Test article absorbance and dilution factor by the product of the antibody's Absorptivity Constant and instrument's path length (for example, but not limited to an amivantamab's Absorptivity Constant of 1.40 (mg/mL)⁻1 cm⁻1 and instrument's path length of 1 cm).

Protein Concentration Results Consistent with Stability

In one embodiment, stability is defined as having a protein concentration of 40 to 60 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 45 to 55 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 43 to 57 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 47 mg/mL to 54 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 45 mg/mL to 55 mg/mL after storage for about 12 months or more and at a temperature of about 5° C., after storage for about 12 months or more and at a temperature of about 25° C., and/or after storage for about 2 years or more and at a temperature of about 5° C.

Analytical Tests—Potency

Potency (Epidermal Growth Factor Receptor (EGFR) Binding)

The in vitro binding of drug product to EGFR is demonstrated using a homogeneous competitive time resolved fluorescence resonance energy transfer (TR-FRET) assay format. In this procedure, varying concentrations of unlabeled bispecific EGFR-cMet antibody sample compete with donor fluorophore (Europium (Eu) chelate) labeled bispecific EGFR-cMet antibody for binding to an acceptor fluorophore (Cy5)-labeled EGFR antigen. Excitation of the donor fluorophore results in a transfer of energy to the bound acceptor fluorophore (FRET process). The resultant FRET is detected by emission of light at 665 nm using a microplate reader capable of measuring time-resolved fluorescence. Sample dose response curves are compared to the RM.

EGFR Binding Materials and Methods. Certified commercial EGFR, a recombinant human EGFR/ErbB1/HER1 with C-terminal His tag is reacted with certified commercial Cy5 Mono NHS Ester to produce Cy5-labeled EGFR. Validated bispecific EGFR-cMet antibody is reacted with certified commercial Europium (Eu) chelate to produce Eu-labeled bispecific EGFR-cMet antibody. Serial dilutions of bispecific EGFR-cMet antibody reference material (RM), assay control and test articles are tested in parallel on the same assay plate. Eu labeled bispecific EGFR-cMet antibody is added to each RM, assay control, and test article followed gentle shaking of the assay plate. Cy5-EGFR is then similarly added, the assay plate again gentle shaken, and incubated in the dark for 4±1 hours. Fluorescence is then measured by spectrophotometry at 665 nm, plotted against antibody concentration and analyzed by a four-parameter logistic model. The antibody concentration required to obtain half of the maximum fluorescence response (EC50) is determined for RM, assay control and samples. The potencies of assay control and samples are calculated based on the ratio of the sample (or control) and RM EC50 values and reported as a percentage activity relative to the RM.

EGFR Binding Activity results consistent with stability. In one embodiment, stability is defined as 50%-150% binding activity relative to Reference Material after storage for about 12 months or more and at a temperature of about 5° C., after storage for about 12 months or more and at a temperature of about 25° C., and/or after storage for about 2 years or more and at a temperature of about 5° C. In one embodiment, stability is defined as 60%-140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined 60%-140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined 65%-130% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging between about 80% to 120% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging between about 70% to 130% binding activity relative to Reference Material after storage for about 12 months or more and at a temperature of about 5° C., after storage for about 12 months or more and at a temperature of about 25° C., and/or after storage for about 2 years or more and at a temperature of about 5° C.

Potency (cMet Binding)

The in vitro binding of bispecific EGFR-cMet antibody to c-MET is demonstrated using a homogeneous competitive time resolved fluorescence resonance energy transfer (TR-FRET) assay format. In this procedure, varying concentrations of unlabeled bispecific EGFR-cMet antibody sample compete with donor fluorophore (Europium (Eu) chelate) labeled bispecific EGFR-cMet antibody for binding to an acceptor fluorophore (Cy5)-labeled c-MET antigen. Excitation of the donor fluorophore results in a transfer of energy to the bound acceptor fluorophore (FRET process). The resultant FRET is detected by emission of light at 665 nm using a microplate reader capable of measuring time-resolved fluorescence. Sample dose response curves are compared to the Reference Material (RM).

c-MET Binding Materials and Methods. Certified commercial cMet, a recombinant cMet/HGFR with c-terminal HIS-tag tag is reacted with certified commercial Cy5 Mono NHS Ester to produce Cy5-labled c-MET. Validated bispecific EGFR-cMet antibody is reacted with certified commercial Europium (Eu) chelate to produce Eu labeled bispecific EGFR-cMet antibody. Serial dilutions of bispecific EGFR-cMet antibody RM, assay control and test articles are tested in parallel on the same assay plate. Eu labeled bispecific EGFR-cMet antibody is added to each RM, assay control, and test article followed by gentle shaking of the assay plate. Cy5-c-MET is then similarly added, the assay plate again gently shaken, and incubated in the dark for 4±1 hours. Fluorescence is then measured by spectrophotometry at 665 nm, plotted against antibody concentration and analyzed by a four-parameter logistic model. The antibody concentration required to obtain half of the maximum fluorescence response (EC50) is determined for RM, assay control and samples. The potencies of assay control and samples are calculated based on the ratio of the sample (or control) and RM EC50 values and reported as a percentage activity relative to the RM.

cMet Binding Activity results consistent with stability. In one embodiment, stability is defined as ranging between about 50% to about 150% binding activity relative to Reference Material after storage for about 12 months or more and at a temperature of about 5° C., after storage for about 12 months or more and at a temperature of about 25° C., and/or after storage for about 2 years or more and at a temperature of about 5° C. In one embodiment, stability is defined as ranging between about 60% to about 140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as ranging between about 60% to 140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as ranging between about 65% to 125% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging about 80% to about 120% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging about 75% to about 125% binding activity relative to Reference Material after storage for about 12 months or more and at a temperature of about 5° C., after storage for about 12 months or more and at a temperature of about 25° C., and/or after storage for about 2 years or more and at a temperature of about 5° C.

Analytical Tests—Surfactant

Polysorbate-80 Quantification

Polysorbate 80 is quantitatively determined by mixed-mode ion-exchange/hydrophobic HPLC.

PS 80 Materials and Methods. Analysis conducted with a gradient HPLC equipped with a 2.1×20 mm on-line column containing a 30 μm water-wetable, mixed-mode polymeric spherical sorbent particles, an ELSD, and a temperature-controlled column compartment at 30° C. The flow rate is set to 1 mL/minute and the ELSD evaporator temperature is set to 50° C. Mobile Phase A is 2% v/v Formic acid in water and Mobile Phase B is 2% v/v Formic acid in Isopropyl alcohol. Neat polysorbate 80 is used to create calibration and check standards. Test article samples are injected neat.

Polysorbate 80 results consistent with stability. In one embodiment, stability is defined as a PS80 concentration of 0.03-0.08% after storage for about 12 months or more and at a temperature of about 5° C., after storage for about 12 months or more and at a temperature of about 25° C., and/or after storage for about 2 years or more and at a temperature of about 5° C. In one embodiment, stability is defined as a PS80 concentration of 0.02-0.09% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as a PS 80 concentration of 0.04-0.08% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as a PS 80 concentration of 0.03-0.08% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as a PS 80 concentration of 0.05-0.08% after storage for about 12 months or more and at a temperature of about 5° C., after storage for about 12 months or more and at a temperature of about 25° C., and/or after storage for about 2 years or more and at a temperature of about 5° C. In a most preferred embodiment, stability is defined as a PS 80 concentration of 0.04-0.08% after storage for about 12 months or more and at a temperature of about 5° C., after storage for about 12 months or more and at a temperature of about 25° C., and/or after storage for about 2 years or more and at a temperature of about 5° C.

Analytical Tests—Routine Characterization

Peptide Map

The purpose of this test is to measure the levels of post-translational modifications, such as oxidation, deamidation, and isomerization, that may be present in the antibody structure. Test articles are enzymatically digested to yield peptide segments. These peptides are then evaluated by Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Peptide Mapping materials and methods. Samples are denatured with 6 M Guanidine, 50 mM Tris pH 8.0, 5 mM EDTA and filtered using 30 kDa centrifugal filter device (flow through discarded). The denatured samples are reduced with 1 M Dithiothreitol (DTT), followed by alkylation with 1 M sodium Iodoacetate, and further treated with DTT to quench the reaction. The reaction mixture is exchanged into digestion buffer (50 mM Tris pH 7.0, with 1 mM CaCl₂)) via Sephadex G-25 columns with separate columns used for blanks, reference material, and test articles. An aliquot of 1 mg/mL Trypsin stock solution is added to the sample in digestion buffer yielding a 20 μL/mL trypsin concentration. The solution is incubated at 37° C. for 2 hours±30 minutes. The trypsinized solution is allowed to cool to room temperature and the enzyme is inactivated with Trifluoroacetic acid. The treated samples are evaluated by Ultra High-Performance Liquid Chromatography Mass Spectroscopy (UPLC-MS) equipped with a Waters Acquity BEH (Ethylene Bridged Hybrid) C18, 2.1×100 mm, 1.7 μm, 130 Å column and an attached auto sampler. Mobile phase A is 0.1% Formic Acid in water Mobile phase B is, 0.1% FA in acetonitrile (mobile phase B). The autosampler is set to 2-8° C., the column is set to 40° C. and the flow rate is set to 500 μL/minute. Eluted peptides were subject to electrospray ionization and detected using a calibrated on-line mass spectrometry.

### Example 1: High-Throughput (HTP) Multi-Factorial Screening Studies

High-throughput (HTP) multi-factorial screening studies were conducted to select combinations of formulation buffer, excipient, polysorbate, and pH. This is achieved by generating multiple test formulations consisting of various combinations of buffers, excipients, polysorbates, and pH values. The test formulations are then artificially stressed and analyzed for surrogate markers of protein destabilization.

In these studies, approximately 2004, of each test formulation were held at 65° C. for 24 hours and then allowed to passively return to ambient room temperature. The temperature equilibrated samples were then spectrophotometrically analyzed to determine the absorbance of the test formulations at 350 nm. Increased absorbance at 350 nm is a widely accepted surrogate correlative attribute of protein destabilization and aggregation. Therefore, thermally stressed test formulations with relatively low absorbance values are considered “stable” formulations with increasing measured absorbance values correlating to relative decreasing stability. The composition of the test formulations and their measured absorbance at 350 nm is shown in table 1 below. The buffer concentration for all test formulation was 20 mM. For context, the typical absorbance value of an empty well (blank) at 350 nm is 0.104 Absorption Units (AU).

TABLE 1
The composition of the test formulations
and their measured absorbance at 350 nm

| | | | | Absorbance |
|---|---|---|---|---|
| Buffer | pH | Surfactant | Excipient | Units at 350 nm |
| Phosphate | 7.0 | 0.04% (w/v) | 8% Sucrose | 1.286 |
| | | Polysorbate | 5% Sorbitol | 1.331 |
| | | 20 | 8% Trehalose | 1.324 |
| | | 0.04% (w/v) | 8% Sucrose | 1.352 |
| | | Polysorbate | 5% Sorbitol | 1.383 |
| | | 80 | 8% Trehalose | 1.357 |
| Histidine | 6.5 | 0.04% (w/v) | 8% Sucrose | 0.222 |
| | | Polysorbate | 5% Sorbitol | 0.260 |
| | | 20 | 8% Trehalose | 0.245 |
| | | 0.04% (w/v) | 8% Sucrose | 0.234 |
| | | Polysorbate | 5% Sorbitol | 0.278 |
| | | 80 | 8% Trehalose | 0.280 |
| | 6 | 0.04% (w/v) | 8% Sucrose | 0.165 |
| | | Polysorbate | 5% Sorbitol | 0.180 |
| | | 20 | 8% Trehalose | 0.181 |
| | | 0.04% (w/v) | 8% Sucrose | 0.168 |
| | | Polysorbate | 5% Sorbitol | 0.195 |
| | | 80 | 8% Trehalose | 0.178 |
| | 5.5 | 0.04% (w/v) | 8% Sucrose | 0.135 |
| | | Polysorbate | 5% Sorbitol | 0.138 |
| | | 20 | 8% Trehalose | 0.137 |
| | | 0.04% (w/v) | 8% Sucrose | 0.131 |
| | | Polysorbate | 5% Sorbitol | 0.131 |
| | | 80 | 8% Trehalose | 0.131 |
| Acetate | 5.5 | 0.04% (w/v) | 8% Sucrose | 0.179 |
| | | Polysorbate | 5% Sorbitol | 0.188 |
| | | 20 | 8% Trehalose | 0.201 |
| | | 0.04% (w/v) | 8% Sucrose | 0.195 |
| | | Polysorbate | 5% Sorbitol | 0.207 |
| | | 80 | 8% Trehalose | 0.208 |
| | 5 | 0.04% (w/v) | 8% Sucrose | 0.141 |
| | | Polysorbate | 5% Sorbitol | 0.146 |
| | | 20 | 8% Trehalose | 0.138 |
| | | 0.04% (w/v) | 8% Sucrose | 0.145 |
| | | Polysorbate | 5% Sorbitol | 0.149 |
| | | 80 | 8% Trehalose | 0.133 |
| | 4.5 | 0.04% (w/v) | 8% Sucrose | 0.143 |
| | | Polysorbate | 5% Sorbitol | 0.150 |
| | | 20 | 8% Trehalose | 0.143 |
| | | 0.04% (w/v) | 8% Sucrose | 0.151 |
| | | Polysorbate | 5% Sorbitol | 0.160 |
| | | 80 | 8% Trehalose | 0.130 |
| Citrate | 4.5 | 0.04% (w/v) | 8% Sucrose | 0.515 |
| | | Polysorbate | 5% Sorbitol | 0.596 |
| | | 20 | 8% Trehalose | 0.632 |
| | | 0.04% (w/v) | 8% Sucrose | 0.673 |
| | | Polysorbate | 5% Sorbitol | 0.779 |
| | | 80 | 8% Trehalose | 0.838 |

Absorbance results were largely driven by pH in a non-linear fashion with the lowest absorbance values seen at pH 5.5 and highest values seen at pH 4.5 and 7.0. However, the chemical properties of the buffer also appeared to play a role. At the same pH of 5.5, histidine showed lower values than acetate. Taking into consideration the pKa of acetate (4.76) and histidine (6.04), histidine would have a stronger buffer capacity than acetate at pH 5.5. The stronger capacity explained the lower absorbance values for histidine versus acetate at the same pH value. This may also explain the difference in result between citrate (pKa 3.09) and acetate (4.76) at pH 4.5. Conversely, phosphate (pKa 6.82) should have had a strong buffering capacity at pH 7.0. However, the phosphate absorbance values were five to ten times higher than most values seen in the study and therefore can be categorically ruled out as a potential buffer system. In a similar manner, citrate absorbance values at pH 4.5 were two to five times higher than most values seen in the study. Formulating with citrate close to its pKa would require a pH value impractical for pharmaceutical applications and therefore can also be categorically ruled out as a potential buffer system.

A consistent, albeit mild, effect of excipient species on absorbance values was seen by examining the study as two polysorbate species arms within the eight buffer/pH value combination arms yielding 16 study arms. From this perspective, sucrose showed the lowest absorbance value versus sorbitol and trehalose in 13 of the 16 arms. Therefore, while sorbitol and trehalose could prove to be acceptable excipients, sucrose could be considered a preferred excipient.

Throughout the study, polysorbate 20 generally showed lower absorbance values than corresponding polysorbate 80 value. For context, the primary role of surfactants in monoclonal antibody formulations, such as polysorbates, is to protect the antibody against mechanical (shaking) stress as opposed to the thermal stress used in this HTP screening study. From this perspective, the differences in absorbance values seen between the two polysorbate species can be considered negligible. However, it is noted that the lowest absorbance values observed in this study contained polysorbate 80.

### Example 2: Polysorbate Concentration Range Shaking & Freeze Thaw Study

This study was conducted to determine the range of polysorbate 80 concentration that stabilizes amivantamab from mechanical, interfacial, and freeze/thaw stress. The study also evaluated the protective properties of polysorbate 80 after storage for 12 months at 5° C.

Six identical sets of test formulation vials were created. Each set contained one vial each of test formulations at low (0.03%), target (0.06%) and high (0.08%) polysorbate 80 concentrations (% w/v). All other formulation components were held constant (50 mg/mL amivantamab, 10 mM Histidine, 8.5% Sucrose, 1 mg/mL methionine, 20 μg/mL ethylenediaminetetraacetic acid (EDTA) at pH 5.7). The formulations were dispensed to a fill volume of 7.5 mL into 8R vials, stoppered, capped, and crimp sealed.

To establish general study baseline data, one set of vials was tested at the start of the study to serve as an untreated, Time Zero Control (T=0).

To evaluate the stabilizing effect of polysorbate 80 against mechanical and interfacial stress, one set of vials was placed horizontally on an orbital shaker and shaken at 250 rpm up to 72 hours under ambient room temperature and light conditions (T72h Shaking). During the same 72-hour period, a second corresponding unshaken control set of vials was held vertically at ambient room and light conditions (T72h Control).

To evaluate the stabilizing effect of aged polysorbate 80 against mechanical and interfacial stress, two sets of vials were held for 12 months at 5° C. Using the methods stated above, one set was shaken for 72 hrs (T12m T72h Shaking), the other set held as a control (T12m T72h Control).

To evaluate the stabilizing effect of polysorbate 80 against freeze/thaw stress, one set of vials was subjected to five (5) freeze/thaw cycles (5×FT) with one cycle defined as freezing to −70° C. followed by passive thawing at ambient room temperature.

As shown in the results below, no substantial differences in attribute values seen after shaking stress versus both T=0 and unshaken control samples. Similar results were seen for samples held for 12 months at 5° C. prior to shaking stress. Also, there were no substantial differences in attribute values seen after freeze/thaw stress versus T=0 controls. Under both shaking stress and freeze/thaw stress, there were no substantial differences in attribute values between the low, target, and high polysorbate 80 samples. This indicated that stable amivantamab formulated with polysorbate 80 over a range of 0.03% (w/v) to 0.08% (w/v) can protect against mechanical, interfacial, and freeze/thaw stress.

Table 2. Results for Shaking and Freeze/Thaw Study.

TABLE 2
Results for Shaking and Freeze/Thaw Study

| | | | | | Particulate Matter | | | | | |
|---|---|---|---|---|---|---|---|---|---|---|
| | Test | | | | (Sub-visible) | | | | | |
| | Formulation | | | | ≥10 μm | ≥25 μm | A280 | SE-HPLC | | |
| Test | (% (w/v) | | | Turbidity | particles/ | particles/ | mg/ | Main | HMWS | LMWS |
| Condition | PS 80) | Color | pH | NTU | vial | vial | mL | Component % | % | % |
| T = 0 | 0.03 | ≤B9, ≤BY7, ≤Y7 | 5.8 | 4.2 | 88 | 30 | 50.0 | 98.8 | 1.1 | 0.0 |
| | 0.06 | ≤B9, ≤BY7, ≤Y7 | 5.8 | 4.5 | 37 | 13 | 50.2 | 98.8 | 1.1 | 0.0 |
| | 0.08 | ≤B9, ≤BY7, ≤Y7 | 5.8 | 4.6 | 38 | 10 | 50.1 | 98.8 | 1.1 | 0.1 |
| T72 h | 0.03 | ≤B9, ≤BY7, ≤Y7 | 5.8 | 4.5 | 125 | 12 | 50.3 | 98.8 | 1.1 | 0.1 |
| Shaking | 0.06 | ≤B9, ≤BY7, ≤Y7 | 5.8 | 4.8 | 70 | 8 | 50.9 | 98.9 | 1.1 | 0.1 |
| | 0.08 | ≤B9, ≤BY7, ≤Y7 | 5.7 | 4.6 | 87 | 23 | 50.4 | 98.8 | 1.1 | 0.1 |
| T72 h | 0.03 | ≤B9, ≤BY7, ≤Y7 | 5.8 | 4.4 | 57 | 20 | 50.1 | 98.8 | 1.1 | 0.1 |
| Control | 0.06 | ≤B9, ≤BY7, ≤Y7 | 5.8 | 4.3 | 42 | 3 | 50.0 | 98.8 | 1.1 | 0.1 |
| | 0.08 | ≤B9, ≤BY7, ≤Y7 | 5.8 | 4.5 | 55 | 17 | 50.0 | 98.8 | 1.1 | 0.1 |
| T12 m | 0.03 | ≤B9, ≤BY7, ≤Y7 | 5.9 | 4.7 | 167 | 23 | 49.9 | 98.6 | 1.2 | 0.2 |
| T72 h | 0.06 | ≤B9, ≤BY7, ≤Y7 | 5.9 | 4.7 | 65 | 0 | 50.3 | 98.7 | 1.2 | 0.2 |
| Shaking | 0.08 | ≤B9, ≤BY7, ≤Y7 | 5.8 | 4.6 | 80 | 18 | 50.5 | 98.6 | 1.2 | 0.2 |
| T12 m | 0.03 | ≤B9, ≤BY7, ≤Y7 | 5.8 | 4.5 | 95 | 12 | 50.3 | 98.7 | 1.2 | 0.2 |
| T72 h | 0.06 | ≤B9, ≤BY7, ≤Y7 | 5.8 | 6.4 | 30 | 3 | 50.3 | 98.7 | 1.2 | 0.2 |
| Control | 0.08 | ≤B9, ≤BY7, ≤Y7 | 5.8 | 6.5 | 35 | 5 | 50.2 | 98.6 | 1.2 | 0.2 |
| 5X F/T | 0.06 | ≤B9, ≤BY7, ≤Y7 | 5.8 | 4.7 | 112 | 7 | 50.4 | 98.8 | 1.1 | 0.0 |

TABLE 3
Results for Shaking and Freeze/Thaw Study

| | | | | | | | cIEF | | | | | |
|---|---|---|---|---|---|---|---|---|---|---|---|---|
| | Test | cSDS (Reduced) | | | cSDS (Non- | | | Sum of | Sum of | | | |
| | Formulation | | AG | | Reduced) | | Main | Acidic | Basic | | EGFR | cMET |
| Test | (% (w/v) | Purity | HC | No new | Purity | No new | Peak | Peaks | Peaks | PS80 | Binding | Binding |
| Condition | PS 80) | % | % | peak >1.0% | % | peak >1.0% | % | % | % | (%) | % | % |
| T0 | 0.03 | 95.4 | 3.9 | NNP | 98.4 | NNP | 74.4 | 23.4 | 2.3 | 0.027 | 106 | 93 |
| | 0.06 | 95.4 | 3.9 | NNP | 98.4 | NNP | 74.7 | 22.3 | 2.9 | 0.055 | 102 | 94 |
| | 0.08 | 95.4 | 3.9 | NNP | 98.3 | NNP | 75.3 | 22.3 | 2.4 | 0.076 | 109 | 100 |
| T72 h | 0.03 | 95.1 | 4.0 | NNP | 98.5 | NNP | 74.1 | 23.3 | 2.7 | 0.027 | 94 | 104 |
| Shaking | 0.06 | 95.3 | 3.9 | NNP | 98.5 | NNP | 73.5 | 23.7 | 2.9 | 0.053 | 110 | 99 |
| | 0.08 | 95.3 | 3.9 | NNP | 98.5 | NNP | 73.6 | 23.4 | 2.9 | 0.074 | 104 | 98 |
| T72 h | 0.03 | 95.3 | 3.9 | NNP | 98.5 | NNP | 73.9 | 23.3 | 2.9 | 0.027 | 93 | 99 |
| Control | 0.06 | 95.3 | 3.9 | NNP | 98.5 | NNP | 73.3 | 23.8 | 2.9 | 0.052 | 104 | 100 |
| | 0.08 | 95.3 | 3.9 | NNP | 98.5 | NNP | 74.7 | 22.9 | 2.5 | 0.074 | 107 | 99 |
| T12 m | 0.03 | 95.2 | 4.0 | NNP | 98.0 | NNP | 74.2 | 23.0 | 2.9 | 0.031 | 92 | 102 |
| T72 h | 0.06 | 95.2 | 4.0 | NNP | 97.9 | NNP | 74.5 | 22.6 | 3.0 | 0.051 | 98 | 96 |
| Shaking | 0.08 | 95.2 | 4.0 | NNP | 98.0 | NNP | 74.4 | 22.9 | 2.6 | 0.076 | 90 | 83 |
| T12 m | 0.03 | 95.2 | 4.0 | NNP | 98.1 | NNP | 74.1 | 22.8 | 3.2 | 0.031 | 88 | 103 |
| T72 h | 0.06 | 95.2 | 4.0 | NNP | 98.0 | NNP | 74.1 | 22.8 | 3.1 | 0.051 | 102 | 98 |
| Control | 0.08 | 95.2 | 4.0 | NNP | 98.1 | NNP | 74.5 | 22.7 | 2.9 | 0.078 | 103 | 81 |
| 5xFT | 0.06 | 95.2 | 4.0 | NNP | 98.5 | NNP | 74.5 | 22.7 | 2.9 | 0.054 | 101 | 104 |

### Example 3: Metal Spiking Study

This study was performed to evaluate the impact of metal ions and peroxides potentially present or introduced during manufacturing processes of bispecific EGFR-cMet antibody. This was designed to assess the formulation stability under a set of exaggerated stress conditions and the efficacy of the supplemental formulation excipients with respect to the reduction or prevention of oxidative pathways.

Test formulations consisted of 50 mg/mL amivantamab, 10 mM Histidine, 8.5% (w/v) Sucrose, 0.06% (w/v) PS80, pH 5.7 supplemented with or without 1 mg/mL L-Methionine and 20 μg/mL EDTA. The exaggerated stress condition was created by spiking test formulations with oxidizing metals (for instance oxidizing metals include, but are not limited to, Iron (Fe3+), Chromium (Cr3+), Copper (Cu2+), Nickel (Ni2+), and Molybdenum (Mo5+)) and/or dissolved Hydrogen Peroxide. The selection of metals is based on the composition of metal alloy components potentially present in manufacturing processes. Hydrogen peroxide is selected due to potential presence of residue material after its use in decontamination of aseptic manufacturing spaces. The concentrations of metals and hydrogen peroxide evaluated are double the highest values that would be seen in commercial GMP Drug Product manufacturing processes. A summary of the test formulations is listed in table 4 below.

TABLE 4
Composition of Test Formulations and Stressors

| Formulation | | | Metal | Hydrogen |
|---|---|---|---|---|
| ID # | Description | Composition of Test Fonnulation | Spike | Peroxide Spike |
| I | No supplements | 10 mM Histidine, 8.5% sucrose, | No | No |
| | (negative control) | 0.06% PS80 | | |
| II | No supplements + | 10 mM Histidine, 8.5% sucrose, | Yes | No |
| | Metal | 0.06% PS80 | | |
| III | No supplements + | 10 mM Histidine, 8.5% sucrose, | No | Yes |
| | Peroxide | 0.06% PS80 | | |
| IV | EDTA/+ Methionine | 10 mM Histidine, 8.5% sucrose, | No | No |
| | (positive control) | 0.06% PS80, 20 μg/mL EDTA, | | |
| | | 1 mg/mL Mediionine | | |
| V | EDTA + Methionine + | 10 mM Histidine, 8.5% sucrose, | Yes | Yes |
| | Metal + Peroxide | 0.06% PS80, 20 μg/mL EDTA, | | |
| | | 1 mg/mL Methionine | | |

Test formulations were aliquoted into 8R vials at a fill volume of 7.5 mL. The vials were stoppered, capped, and crimp sealed. The vials were placed on stability at recommended (5° C.), accelerated (25° C.), and stressed (40° C.) storage conditions. At designated time points, samples were pulled and assayed for oxidation by peptide mapping.

Study Results

At six-months recommended (5° C.) storage conditions, non-supplemented formulations exposed to metals (II) or hydrogen peroxide (III) showed slight to mild increased oxidation versus non-supplemented formulations (I). This indicates that the exaggerated levels of metals and peroxide appears to induce slight to mild oxidation in non-supplemented formulations (I) under recommended storage conditions. However, under the same six-month 5° C. stability conditions, there was no meaningful difference in the oxidation values observed between methionine/EDTA supplemented formulations (IV) and the same supplemented formulation exposed to metals and hydrogen peroxide (V). Further, the supplemented formulation (IV) showed lower oxidation values than non-supplemented formulations exposed to metals (II) or hydrogen peroxide (III). This indicates formulations supplemented with EDTA and methionine were able to mitigate the oxidation induced by exaggerated levels of metals and peroxide under recommended storage conditions.

The same trends were seen at one month stressed (40° C.) and six months accelerated (25° C.) storage conditions, but on a more pronounced level. Non-supplemented formulations exposed to metals (II) or hydrogen peroxide (III) clearly showed increased oxidation versus non-supplemented formulations (I). However, there was no meaningful difference in the oxidation values observed between methionine/EDTA supplemented formulations (IV) and the same supplemented formulation exposed to metals and hydrogen peroxide (V). Further, the supplemented formulation (IV) clearly showed lower oxidation values than non-supplemented formulations exposed to metals (II) or hydrogen peroxide (III). This demonstrates the robustness of EDTA and methionine supplemented formulations to mitigate oxidation induced by exaggerated levels of metals and peroxide under accelerated and stressed storage conditions.

Taken together, this data shows that formulations supplemented with methionine and EDTA are successful in the robust reduction of metal- and peroxide-mediated oxidation.

### Example 4: Formulation Robustness Development

Study Design

This study was performed to examine the effects of multi-factorial varying of formulation component concentration levels of bispecific EGFR-cMet antibody drug product held at recommended (5° C.) and accelerated (25° C.) conditions. The formulation components evaluated were protein concentration, histidine concentration, sucrose concentration, polysorbate 80 concentration, EDTA/methionine concentration, and pH. The ranges of the factor concentrations tested are listed in Table 5.

TABLE 5
Ranges of the factor concentrations tested

| Test Factors | Low | Target | High |
|---|---|---|---|
| Bispecific EGFR-cMet antibody | 44 | 50 | 56 |
| (amivantamab) protein concentration | | | |
| (mg/mL) | | | |
| pH | 5.2 | 5.7 | 6.2 |
| Histidine concentration (mM) | 8 | 10 | 12 |
| Sucrose concentration (% w/v) | 6.8 | 8.5 | 10.2 |
| EDTA concentration (μg/mL) | 16 | 20 | 24 |
| Methionine concentration (mg/mL) | 0.8 | 1 | 1.2 |
| PS80 concentration (% w/v) | 0.036 | 0.06 | 0.084 |

Based on this criterion, JMP® statistical software was used to create a Fractional Factorial Design with center points (Table 6).

TABLE 6
Composition of Test Formulations

| Test | Protein | | Histidine | Sucrose | EDTA | Methionine | PS80 |
|---|---|---|---|---|---|---|---|
| Formulation | (mg/mL) | pH | (mM) | (% w/v) | (μg/mL) | (mg/mL) | (% w/v) |
| 1 | 44 | 5.2 | 8 | 6.8 | 16 | 0.8 | 0.036 |
| 2 | 44 | 6.2 | 8 | 10.2 | 24 | 1.2 | 0.036 |
| 3 | 50 | 5.7 | 10 | 8.5 | 20 | 1 | 0.06 |
| 4 | 44 | 6.2 | 12 | 10.2 | 16 | 0.8 | 0.036 |
| 5 | 56 | 6.2 | 8 | 10.2 | 16 | 0.8 | 0.084 |
| 6 | 50 | 5.7 | 10 | 8.5 | 20 | 1 | 0.06 |
| 7 | 56 | 5.2 | 12 | 6.8 | 24 | 1.2 | 0.084 |
| 8 | 56 | 6.2 | 12 | 6.8 | 24 | 1.2 | 0.036 |
| 9 | 56 | 5.2 | 8 | 10.2 | 16 | 0.8 | 0.036 |
| 10 | 44 | 5.2 | 12 | 10.2 | 24 | 1.2 | 0.084 |
| 11 | 44 | 6.2 | 8 | 6.8 | 16 | 0.8 | 0.084 |

Test formulations were prepared and aliquoted into 8R vials at a fill volume of 7.5 mL. The vials were stoppered, capped, and crimp sealed. The vials were placed on stability at recommended (5° C.) and accelerated (25° C.) conditions. At designated time points, samples were pulled and assayed.

Study Results

The test results for each attribute of the eleven formulations at study initiation (time zero), after 12 months at the recommended storage condition (5° C.), and after 6 months at accelerated temperature (25° C.) are presented in Table 7. The data are reported as the range, mean, and standard deviation of the eleven formulations for each attribute.

The analytical results for all formulations held for 12 months at 5° C. demonstrated little changes in the assay test values indicating stable. The ability for all formulations with multi-variant ranges in excipient concentrations to yield a narrow range of assay test result values demonstrates the robustness of the formulation within the boundaries and storage conditions tested. Additionally, the full range of values observed per assay in this study were consistent with the most preferred embodiment of stability when held at 2-8° C.

The analytical results for all formulations held for six months at accelerated (25° C.) storage conditions showed degradation effects consistent with the stability profile of bispecific EGFR-cMet antibody exposed to prolonged accelerated storage conditions. However, for most results, the magnitude of the effect was relatively minor compared to results seen at 12 months at 5° C. Similarly, the magnitude of increase in the range of result values was also relatively minor with just under half of the of the ranges being equivalent, or less than, those seen at 12 months at 5° C. This demonstrates that even under accelerated storage conditions the multi-variant ranges in excipient concentration resulted in relatively consistent results.

TABLE 7A
Stability Data for bispecific EGFR-cMet antibody Formulations Held at 25°
C. for 6 months and 5° C. for 12 months (Range, Mean, and Standard Deviation)

| | | Time and Storage Conditions | | | | | | | | |
|---|---|---|---|---|---|---|---|---|---|---|
| | | T = 0 M | | | 6 M 25° C. | | | 12 M 5° C. | | |
| Assay | | Range | Mean | SD | Range | Mean | SD | Range | Mean | SD |
| cIEF | % area Main | 73.9-75.2 | 74.5 | 0.4 | 55.1-58.2 | 56.3 | 1.1 | 72.5-74.7 | 73.8 | 0.7 |
| | Peak | | | | | | | | | |
| | % Sum of Acidic | 22-23.4 | 22.7 | 0.4 | 37.8-40.4 | 39.1 | 0.8 | 22.8-24.9 | 23.7 | 0.8 |
| | peaks | | | | | | | | | |
| | % Sum of Basic | 2.4-3.4 | 2.8 | 0.2 | 3.9-5.5 | 4.7 | 0.5 | 2.1-3.0 | 2.5 | 0.3 |
| | peaks | | | | | | | | | |
| cSDS | % Purity | 97.6-98.4 | 98.1 | 0.3 | 95.7-96.5 | 96.1 | 0.3 | 97.8-98.2 | 98.1 | 0.1 |
| | (non-reduced) | | | | | | | | | |
| | % Purity | 95-95.1 | 95.1 | 0.1 | 93-98.3 | 93.2 | 0.1 | 94.8-95.1 | 95.0 | 0.1 |
| | (reduced) | | | | | | | | | |
| SE- | % Aggregate | 0.8-1.0 | 0.9 | 0.1 | 0.8-1.4 | 1.1 | 0.2 | 0.81-1.4 | 1.1 | 0.2 |
| HPLC | % Monomer | 98.9-91.1 | 99.0 | 0.1 | 98-98.6 | 98.3 | 0.2 | 98.5-99 | 98.8 | 0.2 |
| | % Fragment | 0.1-0.1 | 0.1 | 0.0 | 0.5-0.7 | 0.6 | 0.1 | 0.14-0.16 | 0.2 | 0.0 |
| Particulate | Particles/ | 25-215 | 69.9 | 59.2 | 27-97 | 64.9 | 19.5 | 13-165 | 68.0 | 41.5 |
| Matter | container >10 μm | | | | | | | | | |
| (subvis) ^{a} | Particles/ | 5-44 | 15.5 | 11.3 | 5-8 | 11.7 | 4.6 | 0-25 | 10.6 | 8.3 |
| | container >25 μm | | | | | | | | | |
| Turbidity | NTU | 3.5-5.1 | 4.1 | 0.5 | 3.6-5.1 | 4.4 | 0.5 | 3.5-4.9 | 4.1 | 0.5 |

^{a} The high standard deviation is most likely due to assay variability at these low levels of particulate matter

### Example 5: Formulated Drug Bulk Production

Process Description

Processing Solutions

TABLE 7B
Processing Solutions Target Composition and Ranges

| Solution | Composition and Ranges |
|---|---|
| Diafiltration | 10 mM Histidine, 8.5% Sucrose, |
| Buffer | 1 mg/mL L-methionine, pH 5.6 ± 0.3 |
| Polysorbate 80, | 10 mM Histidine, 8.5% Sucrose, |
| EDTA Stock | 1 mg/mL L-methionine, |
| Solution | 6.0% (w/v) Polysorbate 80, |
| | 2 mg/mL EDTA, pH 5.6 ± 0.3 |

Ultrafiltration/Diafiltration (UF/DF)

Ultrafiltration/diafiltration (UF/DF) is performed to re-formulate the amivantamab Virus Retentive filtrate intermediate manufacturing solution to a pre-formulated bulk (pFB) solution consisting of 50 mg/mL amivantamab, 10 mM Histidine, 8.5% Sucrose, 1 mg/mL L-methionine, pH 5.7.

Preparation of Amivantamab Formulated Bulk (FB)

Polysorbate 80 (6.0% w/v) and EDTA (2 mg/mL) stock solution is added to the pFB at a 1:100 dilution to obtain a final concentration of 0.06% (w/v) Polysorbate 80, and 20 μg/mL EDTA yielding the Formulated Bulk (FB) consisting of 50 mg/mL amivantamab in 10 mM Histidine, 8.5% (w/v) Sucrose, 1 mg/mL L-methionine, 0.06% Polysorbate 80, 20 μg/mL EDTA, pH 5.7. The FB solution is then mixed uniformly. Final filtration of the Formulated Bulk is achieved using a sterile 0.45/0.22 μm filter immediately followed with a subsequent, in-line 0.22 μm filter.

Final Bulk Fill

Following final filtration, the FB is filled into polycarbonate Biotainer(s). The fill volume is 20% to 90% of the biotainer's stated volume.

Final Bulk Storage and Shipping

Storage and Shipment Conditions of the Formulated Bulk Prior to Drug Product production is 5° C.±3° C. protected from light if FB is stored for about one week or less or −40° C.±10° C. protected from light if FB is stored for more than one week.

### Example 6: Drug Formulation: Composition and Components of Primary Packaging

Provided herein is a tabular summary of the composition of the Amivantamab Drug Product Formulation (Table 8).

TABLE 8
Composition of Amivantamab Drug Product

| Component | Composition | | Amount per mL | |
|---|---|---|---|---|
| Amivantamab | 50 | mg | 50 | mg |
| L-Histidine | 10 | mM | 0.413 | mg |
| L-Histidine Hydro-chloride | | | 1.538 | mg |
| monohydrate | | | | |
| Sucrose | 8.5% (w/v) | | 85 | mg |
| Polysorbate 80 | 0.06% | | 0.60 | mg |
| L-Methionine | 1.0 | mg/mL | 1.0 | mg |
| EDTA Disodium salt, Dihydrate | 20 | μg/mL | 0.02 | mg |

| | | |
|---|---|---|
| Water for Injection | q.s to 1.0 mL | q.s to 1.0 mL |

Amivantamab drug product (DP) primary packaging consists of a glass vial, a polymer vial stopper, and an aluminum seal. Tables 9 list specific components for the primary packaging material.

TABLE 9
Primary packaging material components

| Component | Description |
|---|---|
| Glass vial | 8 mL glass Type 1 borosilicate |
| Stopper | 20 mm butyl rubber, FluroTec coated stopper |
| Seals | 20 mm aluminum seal with Flip-Off button |

### Example 7: Description of Stability Study

This study was conducted to monitor amivantamab Drug Product attributes placed on stability under various environmental conditions and lengths of time. Study test articles were prepared by aliquoting Formulated Bulk into 8R vials at a fill volume of 7.5 mL. The vials were stoppered, capped, and crimp sealed

All studies were to be performed with vials in an inverted orientation.

TABLE 10
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

Stability Study Results

The stability results for amivantamab DP held under recommended, accelerated, and stressed conditions are listed below. At all-time points for DP held at recommended storage conditions, all test parameter result values observed per assay study were consistent with or exceeded the criteria consistent with the most preferred embodiment of the stability when held after storage for about 12 months or more and at a temperature of about 5° C. or for about 24 or more and at a temperature of about 5° C. DP held at accelerated conditions (25° C.) for 12 months showed results consistent with or exceeding either the most preferred or the preferred embodiments of the stability after storage for about 12 months or more and at a temperature of about 25° C., and/or after storage for about 2 years or more at a temperature of about 5° C. Similarly, peptide map results showed little to no consequential change over time in the measured percent of post translational modification.

Results for amivantamab DP held at accelerated and stressed conditions showed the expected rates of degradation for Drug Product exposed to prolonged accelerated and stressed storage conditions. Of particular note, DP held at accelerated conditions (25° C.) for 12 months showed results consistent with or exceeding either the most preferred or the preferred embodiments of the stability.

### 5° C. Data

TABLE 11
Stability Results for Amivantamab Drug Product Stored at 5° C.

| | | | | | | cSDS (Reduced) | | |
|---|---|---|---|---|---|---|---|---|
| | | | | | | | | new |
| | | | | Particulate Matter | | | | peaks, as |
| | | | | Sub-visible | | | | compared |
| | | | | ≥10 μm: | ≥25 μm: | | AG | to |
| | Color of | | Turbidity | particles | particles | Purity: | HC: | Reference |
| Months | Solution | pH | (NTU) | per vial | per vial | % | % | Material |
| 0 | ≤BY6, ≤B7, | 5.8 | 4.3 | 65 | 2 | 95.1 | 4.2 | No new |
| | ≤Y6 | | | | | | | peak >1.0% |
| 3 | ≤BY7, ≤B8, | 5.7 | 4.4 | 71 | 1 | 94.9 | 4.3 | No new |
| | ≤Y7 | | | | | | | peak >1.0% |
| 6 | ≤BY7, ≤B9, | 5.7 | 4.1 | 20 | 0 | 95.2 | 4.1 | No new |
| | ≤Y7 | | | | | | | peak >1.0% |
| 9 | ≤BY7, ≤B7, | 5.9 | 4.0 | 89 | 5 | 95.0 | 4.2 | No new |
| | ≤Y7 | | | | | | | peak >1.0% |
| 12 | ≤BY7, ≤B8, | 5.5 | 4.4 | 23 | 1 | 95.1 | 4.1 | No new |
| | ≤Y7 | | | | | | | peak >1.0% |
| 18 | ≤BY7, ≤B8, | 5.7 | 4.2 | 71 | 2 | 95.1 | 4.1 | No new |
| | ≤Y7 | | | | | | | peak >1.0% |
| 24 | ≤BY7, ≤B8, | 5.6 | 4.1 | 16 | 1 | 94.9 | not | No new |
| | ≤Y7 | | | | | | tested | peak >1.0% |
| 36 | | | | | | | | |

| | | | SE HPLC | | | Protein |
|---|---|---|---|---|---|---|
| | cSDS (Non-reduced) | | Main | | | Conc. by |
| | Purity | | Component | HMWS | LMWS | A₂₈₀ |
| Months | (%) | New Peaks (%) | (%) | (%) | (%) | (mg/mL) |
| 0 | 98.3 | No new peak >1.0% | 99.2 | 0.8 | <0.1 | 53.5 |
| | | compared to | | | | |
| | | Reference Material | | | | |
| 3 | 98.2 | No new peak >1.0% | 99.1 | 0.8 | <0.1 | 53.4 |
| | | compared to | | | | |
| | | Reference Material | | | | |
| 6 | 98.1 | No new peak >1.0% | 99.1 | 0.8 | <0.1 | 53.1 |
| | | compared to | | | | |
| | | Reference Material | | | | |
| 9 | 98.2 | No new peak >1.0% | 99.1 | 0.8 | <0.1 | 53.3 |
| | | compared to | | | | |
| | | Reference Material | | | | |
| 12 | 98.1 | No new peak >1.0% | 99.1 | 0.8 | <0.1 | 53.9 |
| | | compared to | | | | |
| | | Reference Material | | | | |
| 18 | 98.0 | No new peak >1.0% | 99.0 | 0.9 | 0.1 | 53.7 |
| | | compared to | | | | |
| | | Reference Material | | | | |
| 24 | 98.1 | No new peak >1.0% | 98.9 | 1.0 | not tested | 52.8 |
| | | compared to | | | | |
| | | Reference Material | | | | |
| 36 | | | | | | |

| | cIEF | | | EGFR Binding | cMET Binding | |
|---|---|---|---|---|---|---|
| | | Sum of | Sum of | activity relative | activity relative | Polysorbate |
| | Main | acidic | Basic | to Reference | to Reference | 80 |
| Months | peak (%) | peaks (%) | peaks (%) | Material (%) | Material (%) | (%) |
| 0 | 76 | 22 | <3.0 | 108 | 103 | 0.0561 |
| 3 | 75 | 22 | <3.0 | 107 | 104 | 0.0563 |
| 6 | 75 | 22 | <3.0 | 127 | 104 | 0.0578 |
| 9 | 74 | 23 | <3.0 | 92 | 98 | 0.0560 |
| 12 | 74 | 24 | 2 | 95 | 96 | 0.0597 |
| 18 | 74 | 23 | 3 | 110 | 100 | 0.0577 |
| 24 | 73 | 24 | 3 | 101 | 93 | 0.0603 |
| 36 | | | | | | |

| | Post Translational Modification- Oxidation Site | | | | | |
|---|---|---|---|---|---|---|
| | anti-EGFR | anti-EGFR | anti-EGFR | anti-EGFR | anti-EGFR | anti-EGFR |
| | HC Met 34*/ | HC Met 103*/ | HC Met 108*/ | HC Met 260/ | HC Met 436/ | n/a/anti-c-Met |
| | anti-c-Met | anti-c-Met | anti-c-Met | anti-c-Met | anti-c-Met | LC Trp 32*, |
| | n/a | n/a | n/a | HC Met 254 | HC Met 430 | Trp 35 |
| Months | (%) | (%) | (%) | (%) | (%) | (%) |
| 0 | 0.2 | 1.6 | 2.2 | 2.3 | 0.7 | No result |
| | | | | | | reposted |
| 3 | 0.2 | 1.2 | 2.4 | 2.1 | 0.7 | 1.5 |
| 6 | 0.2 | 1.5 | 1.7 | 2.2 | 0.7 | 1.7 |
| 9 | 0.2 | 1.2 | 3.2 | 2.4 | 0.8 | 2 |
| 12 | 0.4 | 1.8 | 1.1 | 2.8 | 1.1 | 1.1 |
| 18 | 0.3 | 1.7 | 1.8 | 2.5 | 1.0 | 1.2 |
| 24 | 1.0 | 2.4 | 2.5 | 3.3 | 1.5 | 1.5 |
| 36 | | | | | | |

| | Post Translational Modification- Deamidation Site | | |
|---|---|---|---|
| | | anti-EGFR | |
| | anti-EGFR | HC Asn 392/ | anti-EGFR |
| | HC Asn 333*/ | anti-c-Met | n/a/ |
| | anti-c-Met | HC Asn 386, | anti-c-Met |
| | HC Asn 327 | Asn 391 | HC Asn 55*, 59* |
| Months | (%) | (%) | (%) |
| 0 | 0.7 | 2.7 | 4.4 |
| 3 | 0.7 | 2.2 | 3.7 |
| 6 | 0.7 | 2.6 | 3.6 |
| 9 | 0.8 | 2 | 4.4 |
| 12 | 0.8 | 2.8 | 3.7 |
| 18 | 0.9 | 3.1 | 3.7 |
| 24 | Not tested | 3.0 | 4.2 |
| 36 | | | |

Post Translational Modification- Isomerization Site

| | anti-EGFR | anti-EGFR |
|---|---|---|
| | HC Asp 53*, Asp 54*/ | HC Asp 99*/ |
| | anti-c-Met | anti-c-Met |
| | n/a | n/a |
| Months | (%) | (%) |
| 0 | nr | 1 |
| 3 | 0.4 | 1 |
| 6 | 0.5 | 1.2 |
| 9 | 0.5 | 1.3 |
| 12 | 0.8 | 1.2 |
| 18 | 0.3 | 1.6 |
| 24 | 0.5 | 1.6 |
| 36 | | |

### 25° C. Data

TABLE 12
Stability Results for Amivantamab Drug Product Stored at 25° C.

| Particulate Matter | |
|---|---|
| Sub-visible | cSDS (Reduced) |

| | | | | ≥10 μm: | ≥25 μm: | | AG | |
|---|---|---|---|---|---|---|---|---|
| | Color of | | Turbidity | particles | particles | Purity | HC: | |
| Months | Solution | pH | (NTU) | per vial | per vial | % | % | new peaks |
| 0 | ≤BY6, ≤B7, | 5.8 | 4.3 | 65 | 2 | 95.1 | 4.2 | No new peak >1.0% |
| | ≤Y6 | | | | | | | compared to |
| | | | | | | | | Reference Material |
| 3 | ≤BY6, ≤B7, | 5.7 | 4.3 | 32 | 0 | 94.6 | 4.1 | No new peak >1.0% |
| | ≤Y6 | | | | | | | compared to |
| | | | | | | | | Reference Material |
| 6 | ≤BY7, ≤B7, | 5.8 | 4 | 13 | 0 | 93.9 | 4.1 | No new peak >1.0% |
| | ≤Y7 | | | | | | | compared to |
| | | | | | | | | Reference Material |
| 9 | ≤BY7, ≤B8, | 5.7 | 4.1 | 51 | 1 | 92.7 | 4.2 | No new peak >1.0% |
| | ≤Y7 | | | | | | | compared to |
| | | | | | | | | Reference Material |
| 12 | ≤BY6, ≤B6, | 5.7 | 5 | 40 | 3 | 91.7 | 4.1 | No new peak >1.0% |
| | ≤Y6 | | | | | | | compared to |
| | | | | | | | | Reference Material |

| | cSDS (Non-reduced) | | SE HPLC | | | Protein |
|---|---|---|---|---|---|---|
| | | New | Main | | | Conc. by |
| | Purity | Peaks | Component | HMWS | LMWS | A₂₈₀ |
| Months | (%) | (%) | (%) | (%) | (%) | (mg/mL) |
| 0 | 98.3 | No new peak >1.0% | 99.2 | 0.8 | <0.1 | 53.5 |
| | | compared to | | | | |
| | | Reference Material | | | | |
| 3 | 97.4 | No new peak >1.0% | 98.9 | 0.9 | 0.2 | 52.1 |
| | | compared to | | | | |
| | | Reference Material | | | | |
| 6 | 96.2 | No new peak >1.0% | 98.5 | 1.0 | 0.5 | 53.2 |
| | | compared to | | | | |
| | | Reference Material | | | | |
| 9 | 95.5 | No new peak >1.0% | 98.3 | 1.0 | 0.7 | 53.1 |
| | | compared to | | | | |
| | | Reference Material | | | | |
| 12 | 94.5 | No new peak >1.0% | 98.0 | 1.1 | 0.9 | 53.7 |
| | | compared to | | | | |
| | | Reference Material | | | | |

| | cIEF | | | EGFR Binding | cMET Binding | |
|---|---|---|---|---|---|---|
| | | Sum of | Sum of | activity relative | activity relative | Polysorbate |
| | Main | acidic peaks | Basic peaks | to Reference | to Reference | 80 |
| Months | peak (%) | (%) | (%) | Material (%) | Material (%) | (%) |
| 0 | 76 | 22 | 2 | 108 | 103 | 0.0561 |
| 3 | 64 | 32 | 4 | 93 | 101 | 0.0552 |
| 6 | 56 | 39 | 5 | 82 | 96 | 0.0550 |
| 9 | 52 | 43 | 5 | 86 | 96 | 0.0578 |
| 12 | 46 | 48 | 6 | 79 | 89 | 0.055 |

Post Translational Modification- Oxidation Site

| | | | | | | anti-EGFR |
|---|---|---|---|---|---|---|
| | anti-EGFR | anti-EGFR | anti-EGFR | anti-EGFR | anti-EGFR | n/a/ |
| | HC Met 34*/ | HC Met 103*/ | HC Met 108*/ | HC Met 260/ | HC Met 436/ | anti-c-Met |
| | anti-c-Met | anti-c-Met | anti-c-Met | anti-c-Met | anti-c-Met | LC Trp 32*, |
| | n/a | n/a | n/a | HC Met 254 | HC Met 430 | Trp 35 |
| Months | (%) | (%) | (%) | (%) | (%) | (%) |
| 0 | 0.2 | 1.6 | 2.2 | 2.3 | 0.7 | No result reposted |
| 3 | 0.5 | 1.7 | 3.1 | 2.7 | 1.2 | 2.5 |
| 6 | 0.4 | 2.2 | 1.9 | 2.6 | 0.8 | 4.1 |
| 9 | 0.5 | 2.3 | 1.1 | 2.9 | 1.2 | 3.2 |
| 12 | 0.4 | 2.4 | 1.3 | 2.9 | 1.2 | 4.0 |

| | Post Translational Modification- Deamidation Site | | |
|---|---|---|---|
| | | anti-EGFR | |
| | anti-EGFR | HC Asn 392/ | anti-EGFR |
| | HC Asn 333*/ | anti-c-Met | n/a/ |
| | anti-c-Met | HC Asn 386, | anti-c-Met |
| | HC Asn 327 | Asn 391 | HC Asn 55*, 59* |
| Months | (%) | (%) | (%) |
| 0 | 0.7 | 2.7 | 4.4 |
| 3 | 2.8 | 3.6 | 4.8 |
| 6 | 6.3 | 3.6 | 5.4 |
| 9 | Not tested | 3.9 | 6.8 |
| 12 | Not Tested | 4.5 | 7.8 |

Post Translational Modification- Isomerization Site

| | anti-EGFR | |
|---|---|---|
| | HC Asp 53*, | anti-EGFR |
| | Asp 54*/ | HC Asp 99*/ |
| | anti-c-Met | anti-c-Met |
| | n/a | n/a |
| Months | (%) | (%) |
| 0 | nr | 1 |
| 3 | 0.7 | 3.6 |
| 6 | 1.3 | 1.8 |
| 9 | 2.7 | 6.1 |
| 12 | 3.1 | 7.4 |

### 40° C. Data

TABLE 13
Stability Results for Amivantamab Drug Product Stored at 40° C.

| | |
|---|---|
| Particulate Matter | |
| Sub-visible | cSDS (Reduced) |

| | | | | ≥10 μm: | ≥25 μm: | | AG | |
|---|---|---|---|---|---|---|---|---|
| | Color of | | Turbidity | particles | particles | Purity: | HC: | |
| Months | Solution | pH | (NTU) | per vial | per vial | % | % | new peaks |
| 0 | ≤BY6, ≤B7, | 5.8 | 4.3 | 65 | 2 | 95.1 | 4.2 | No new peak >1.0% |
| | ≤Y6 | | | | | | | compared to |
| | | | | | | | | Reference Material |
| 1 | ≤BY6, ≤B6, | 5.8 | 5.2 | 167 | 6 | 92.2 | 4.3 | No new peak >1.0% |
| | ≤Y6 | | | | | | | compared to |
| | | | | | | | | Reference Material |
| 3 | ≤BY6, ≤B6, | 5.7 | 4.9 | 32 | 1 | 88.4 | 4.2 | No new peak >1.0% |
| | ≤Y6 | | | | | | | compared to |
| | | | | | | | | Reference Material |
| 6 | ≤BY6, ≤B6, | 5.7 | 5 | 28 | 1 | 82.7 | 4.4 | Peak 2: |
| | ≤Y6 | | | | | | | 1.032% PI 12.82 |

| | | | SE HPLC | | | Protein |
|---|---|---|---|---|---|---|
| | cSDS (Non-reduced) | | Main | | | Conc. by |
| | Purity | | Component | HMWS | LMWS | A₂₈₀ |
| Months | (%) | New Peaks | (%) | (%) | (%) | (mg/mL) |
| 0 | 98.3 | No new peak >1.0% | 99.2 | 0.8 | <0.1 | 53.5 |
| | | compared to | | | | |
| | | Reference Material | | | | |
| 1 | 95.5 | No new peak >1.0% | 98.4 | 1.0 | 0.4 | 52.7 |
| | | compared to | | | | |
| | | Reference Material | | | | |
| 3 | 90.7 | No new peak >1.0% | 96.5 | 1.9 | 1.6 | 53.7 |
| | | compared to | | | | |
| | | Reference Material | | | | |
| 6 | 82.4 | Peak 1: 1.19% PI | 91.5 | 3.9 | 4.6 | 53.3 |
| | | 12.89 | | | | |

| | cIEF | | | EGFR Binding | cMET Binding | |
|---|---|---|---|---|---|---|
| | | Sum of | Sum of | activity relative | activity relative | Polysorbate |
| | Main | acidic | Basic | to Reference | to Reference | 80 |
| Months | peak (%) | peaks (%) | peaks (%) | Material (%) | Material (%) | (%) |
| 0 | 76 | 22 | <3.0 | 108 | 103 | 0.0561 |
| 1 | 46 | 47 | 6 | 99 | 97 | 0.0560 |
| 3 | 23 | 73 | 5 | 63 | 91 | 0.0541 |
| 6 | 6 | 93 | <3.0 | 33 | 74 | 0.0543 |

| | Post Translational Modification- Oxidation Site | | | | | |
|---|---|---|---|---|---|---|
| | | | | | | anti-EGFR |
| | anti-EGFR | anti-EGFR | anti-EGFR | anti-EGFR | anti-EGFR | n/a/ |
| | HC Met 34*/ | HC Met 103*/ | HC Met 108*/ | HC Met 260/ | HC Met 436/ | anti-c-Met |
| | anti-c-Met | anti-c-Met | anti-c-Met | anti-c-Met | anti-c-Met | LC Trp 32*, |
| | n/a | n/a | n/a | HC Met 254 | HC Met 430 | Trp 35 |
| Months | (%) | (%) | (%) | (%) | (%) | (%) |
| 0 | 0.2 | 1.6 | 2.2 | 2.3 | 0.7 | No result |
| | | | | | | reposted |
| 1 | 0.4 | 1.6 | 3.1 | 2.8 | 0.9 | 1.8 |
| 3 | 0.6 | 2.5 | 4.5 | 3.4 | 1.4 | 7.7 |
| 6 | 0.4 | 7 | 5.1 | 4 | 1.4 | 18.8 |

| | Post Translational Modification- Deamidation Site | | |
|---|---|---|---|
| | | anti-EGFR | |
| | anti-EGFR | HC Asn 392/ | anti-EGFR |
| | HC Asn 333*/ | anti-c-Met | n/a/ |
| | anti-c-Met | HC Asn 386, | anti-c-Met |
| | HC Asn 327 | Asn 391 | HC Asn 55*, 59* |
| Months | (%) | (%) | (%) |
| 0 | 0.7 | 2.7 | 4.4 |
| 1 | 10.8 | 4.3 | 5.8 |
| 3 | 27.9 | 6.2 | 10.2 |
| 6 | 51.5 | 9.5 | 15.9 |

Post Translational Modification- Isomerization Site

| | anti-EGFR | |
|---|---|---|
| | HC Asp 53*, | anti-EGFR |
| | Asp 54*/ | HC Asp 99*/ |
| | anti-c-Met | anti-c-Met |
| | n/a | n/a |
| Months | (%) | (%) |
| 0 | nr | 1 |
| 1 | 2.2 | 5.6 |
| 3 | 4.5 | 21 |
| 6 | 6.5 | 18.3 |

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 14
Sequences

| SEQ ID NO: | Sequence |
|---|---|
| SEQ ID NO: 1 | TYGMH |
| HCDR1, EGFR binding | |
| arm (Kabat method) | |
| SEQ ID NO: 2 | VIWDDGSYKYYGDSVKG |
| HCDR2, EGFR binding | |
| arm (Kabat method) | |
| SEQ ID NO: 3 | DGITMVRGVMKDYFDY |
| HCDR3, EGFR binding | |
| arm (Kabat method) | |
| SEQ ID NO: 4 LCDR1, | RASQDISSALV |
| EGFR binding arm (Kabat | |
| method) | |
| SEQ ID NO: 5 LCDR2, | DASSLES |
| EGFR binding arm (Kabat | |
| method) | |
| SEQ ID NO: 6 LCDR3, | QQFNSYPLT |
| EGFR binding arm (Kabat | |
| method) | |
| SEQ ID NO: 7 HCDR1, c- | SYGIS |
| Met binding arm (Kabat | |
| method) | |
| SEQ ID NO: 8 HCDR2, c- | WISAYNGYTNYAQKLQG |
| Met binding arm (Kabat | |
| method) | |
| SEQ ID NO: 9 HCDR3, c- | DLRGTNYFDY |
| Met binding arm (Kabat | |
| method) | |
| SEQ ID NO: 10 LCDR1, c- | RASQGISNWLA |
| Met binding arm (Kabat | |
| method) | |
| SEQ ID NO: 11 LCDR2, c- | AASSLLS |
| Met binding arm (Kabat | |
| method) | |
| SEQ ID NO: 12 LCDR3, c- | QQANSFPIT |
| Met binding arm (Kabat | |
| method) | |
| SEQ ID NO: 13 | QVQLVESGGGVVQPGRSLRLSCAASGFTFSTYGMHWVRQAPGKGLEWV |
| VH1, EGFR binding arm | AVIWDDGSYKYYGDSVKGRFTISRDNSKNTLYL |
| | QMNSLRAEDTAVYYCARDGITMVRGVMKDYFDYWGQGTLVTVSS |
| SEQ ID NO: 14 | AIQLTQSPSSLSASVGDRVTITCRASQDISSALVWYQQKPGKAPKLLIYDA |
| VL1, EGFR binding arm | SSLESGVPSRFSGSESGTDFTLTISSLQPEDFATYY |
| | CQQFNSYPLTFGGGTKVEIK |
| SEQ ID NO: 15 | QVQLVQSGAEVKKPGASVKVSCETSGYTFTSYGISWVRQAPGHGLEWM |
| VH2, c-Met binding arm | GWISAYNGYTNYAQKLQGRVTMTTDTSTSTAYM |
| | ELRSLRSDDTAVYYCARDLRGTNYFDYWGQGTLVTVSS |
| SEQ ID NO: 16 | DIQMTQSPSSVSASVGDRVTITCRASQGISNWLAWFQHKPGKAPKLLIYA |
| VL2, c-Met binding arm | ASSLLSGVPSRFSGSGSGTDFTLTISSLQPEDFATYY |
| | CQQANSFPITFGQGTRLEIK |
| SEQ ID NO: 17 HC1 | QVQLVESGGGVVQPGRSLRLSCAASGFTFSTYGMHWVRQAPGKGLEWV |
| | AVIWDDGSYKYYGDSVKGRFTISRDNSKNTLYLQMNSLRAEDTAVYYCA |
| | RDGITMVRGVMKDYFDYWGQGTLVTVSSASTKGPSVFPLAPSSKSTSGG |
| | TAALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPELLGGPS |
| | VFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISK |
| | AKGQPREPQVYTLPPSREEMTKNQVSLTCLVKGFYPSDIAVEWESNGQPE |
| | NNYKTTPPVLDSDGSFLLYSKLTVDKSRWQQGNVFSCSVMHEALHNHYT |
| | QKSLSLSPGK |
| SEQ ID NO: 18 LC1 | AIQLTQSPSSLSASVGDRVTITCRASQDISSALVWYQQKPGKAPKLLIYDA |
| | SSLESGVPSRFSGSESGTDFTLTISSLQPEDFATYYCQQFNSYPLTFGGGTK |
| | VEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| SEQ ID NO: 19 HC2 | QVQLVQSGAEVKKPGASVKVSCETSGYTFTSYGISWVRQAPGHGLEWM |
| | GWISAYNGYTNYAQKLQGRVTMTTDTSTSTAYMELRSLRSDDTAVYYC |
| | ARDLRGTNYFDYWGQGTLVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSRLTVDKSRWQQGNVFSCSVMHEALHNHYTQKSLSLS |
| | PGK |
| SEQ ID NO: 20 LC2 | DIQMTQSPSSVSASVGDRVTITCRASQGISNWLAWFQHKPGKAPKLLIYA |
| | ASSLLSGVPSRFSGSGSGTDFTLTISSLQPEDFATYYCQQANSFPITFGQGT |
| | RLEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLS |
| | SPVTKSFNRGEC |

What is claimed:

1. A stable aqueous pharmaceutical composition comprising:
a) about 44 mg/mL to about 56 mg/mL of a bispecific epidermal growth factor receptor (EGFR)/hepatocyte growth factor receptor (c-Met) antibody, the bispecific antibody comprising:
a first heavy chain (HC1) comprising a HC1 variable region 1 (VH1);
a first light chain (LC1) comprising a light chain variable region 1 (VL1);
a second heavy chain (HC2) comprising a HC2 variable region 2 (VH2); and
a second light chain (LC2) comprising a light chain variable region 2 (VL2),
wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively, the VH2 comprises the HCDR1, the HCDR2 and the HCDR3 amino acid sequences of SEQ ID NOs: 7, 8 and 9, respectively; and
the VL2 comprises the LCDR1, the LCDR2 and the LCDR3 amino acid sequences of SEQ ID NOs: 10, 11 and 12, respectively;
b) about 8 mM to about 12 mM of histidine and/or pharmaceutically acceptable histidine salt,
c) about 6.8% (w/v) to about 10.2% (w/v) of sucrose,
d) about 0.036% (w/v) to about 0.084% (w/v) of polysorbate 80 (PS80),
e) about to 0.8 mg/mL to about 1.2 mg/mL of methionine,
f) about 16 μg/mL to about 24 μg/mL of ethylenediaminetetraacetic acid (EDTA); and
g) a pH from about 5.2 to about 6.2.

2. The stable aqueous pharmaceutical composition of claim 1, wherein the bispecific EGFR-cMet antibody comprises an HC1 variable region comprising the amino acid sequence of SEQ ID NO:13 and a LC1 variable region comprising the amino acid sequence of SEQ ID NO:14.

3. The stable aqueous pharmaceutical composition of claim 1, wherein the bispecific EGFR-cMet antibody comprises a HC2 variable region comprising the amino acid sequence of SEQ ID NO:15 and a LC2 variable region comprising the amino acid sequence of SEQ ID NO:16.

4. The stable aqueous pharmaceutical composition of claim 1, wherein the HC1 comprises the amino acid sequence of SEQ ID NO:17 and the LC1 comprises the amino acid sequence of SEQ ID NO:18.

5. The stable aqueous pharmaceutical composition of claim 1, wherein the HC2 comprises the amino acid sequence of SEQ ID NO:19 and the LC2 comprises the amino acid sequence of SEQ ID NO:20.

6. The stable aqueous pharmaceutical composition of claim 1, wherein the bispecific EGFR-cMet antibody is amivantamab.

7. The stable aqueous pharmaceutical composition of claim 1, wherein the bispecific EGFR-cMet antibody has a concentration of about 50 mg/mL.

8. The stable aqueous pharmaceutical composition of claim 1, wherein the histidine and/or pharmaceutically acceptable histidine salt has a concentration of about 10 mM.

9. The stable aqueous pharmaceutical composition of claim 1, wherein the histidine and/or pharmaceutically acceptable histidine salt comprises L-histidine and L-histidine hydrochloride monohydrate.

10. The stable aqueous pharmaceutical composition of claim 1, comprising about 8.5% (w/v) sucrose.

11. The stable aqueous pharmaceutical composition of claim 1, comprising about 0.06% (w/v) PS80.

12. The stable aqueous pharmaceutical composition of claim 1, wherein the methionine has a concentration of about 1 mg/mL.

13. The stable aqueous pharmaceutical composition of claim 1, wherein the EDTA has a concentration of about 20 μg/mL.

14. The stable aqueous pharmaceutical composition of claim 1, wherein the pH is about 5.7.

15. The stable aqueous pharmaceutical composition of claim 1, comprising 50 mg/mL of the bispecific EGFR-cMet antibody, 10 mM histidine and/or pharmaceutically acceptable histidine salt, 8.5% (w/v) sucrose, 0.06% (w/v) PS80, 1 mg/mL methionine, and 20 μg/mL EDTA.

16. The stable aqueous pharmaceutical composition of claim 1, wherein the stable aqueous pharmaceutical composition is stable at a temperature of about 2-8° C. for at least two years.

17. The stable aqueous pharmaceutical composition of claim 1, wherein stability is defined based on color of solution, pH, turbidity, number of subvisible particles, percentage of aglycosylated heavy chain (AGHC), percentage of new peak(s), percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, percentage of PS80, or any combination thereof.

18. The stable aqueous pharmaceutical composition of claim 1, wherein the total volume of the composition ranges from about 5 mL to about 10 mL.

19. A method of treating cancer in a subject in need thereof, the method comprising administering to the subject the pharmaceutical composition of claim 1.

20. The method of claim 19, wherein the administering is intravenous.

21. A method for preparing a stable aqueous pharmaceutical composition of a bispecific antibody targeting EGFR and cMet, the bispecific antibody targeting EGFR and cMet comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively; the method comprising: combining a composition comprising about 50 mg/mL of the bispecific antibody, about 10 mM histidine and/or pharmaceutically acceptable histidine salt, about 8.5% Sucrose, and about 1 mg/mL L-methionine with polysorbate 80 to a final concentration of about 0.06% (w/v) and EDTA to a final concentration of about 20 μg/mL, wherein the stable aqueous pharmaceutical composition has about pH 5.7.

22. The method of claim 21, wherein the bispecific EGFR-cMet antibody comprises an HC1 variable region comprising the amino acid sequence of SEQ ID NO:13 and a LC1 variable region comprising the amino acid sequence of SEQ ID NO:14.

23. The method of claim 21, wherein the bispecific EGFR-cMet antibody comprises a HC2 variable region comprising the amino acid sequence of SEQ ID NO:15 and a LC2 variable region comprising the amino acid sequence of SEQ ID NO:16.

24. The method of claim 21, wherein the antibody comprises a heavy chain 1 (HC1) comprising the amino acid sequence of SEQ ID NO:17 and a light chain 1 (LC1) comprising the amino acid sequence of SEQ ID NO:18.

25. The method of claim 21, wherein the antibody comprises a HC2 comprising the amino acid sequence of SEQ ID NO:19 and a LC2 comprising the amino acid sequence of SEQ ID NO:20.

26. The method of claim 21, wherein the antibody is amivantamab.

27. A kit comprising the stable aqueous pharmaceutical composition of claim 1 and instructions for use thereof.

28. An article of manufacture comprising a container holding a stable aqueous pharmaceutical composition in accordance with claim 1.

29. The article of manufacture according to claim 28, wherein the container is a vial with a stopper pierceable by a syringe.

30. A pharmaceutical composition of claim 1 for use in the treatment of cancer.

31. A pharmaceutical composition of claim 1 for use in the preparation of a medicine.

32. Use of a pharmaceutical composition for treating cancer in a subject in need thereof by administering the pharmaceutical composition of claim 1.

33. Use of a pharmaceutical composition according to claim 32, wherein the administration is intravenous.
